(19) United States
(12) Patent Application Publication
(10) Pub. No.: US 2015/0374815 A1
(43) Pub. Date: Dec. 31, 2015

(54) METHODS AND COMPOSITIONS FOR TREATMENT WITH SYNTHETIC NANOCARRIERS AND IMMUNE CHECKPOINT INHIBITORS

(71) Applicant: Selecta Biosciences, Inc., Watertown, MA (US)

(72) Inventors: Takashi Kei Kishimoto, Lexington, MA (US); Petr IIyinskii, Cambridge, MA (US)

(73) Assignee: Selecta Biosciences, Inc., Watertown, MA (US)

(21) Appl. No.: 14/751,106

(22) Filed: Jun. 25, 2015

Related U.S. Application Data
(60) Provisional application No. 62/017,101, filed on Jun. 25, 2014.

(51) Int. Cl. A61K 39/385; C12N 7/00; A61K 39/00; A61K 39/39; A61K 39/12

(52) U.S. Cl. CPC A61K 39/385; A61K 39/39; A61K 39/12; A61K 39/0011; C12N 7/00; A61K 2039/60; A61K 2039/545; A61K 2039/54; A61K 2039/6093; C12N 2710/20034; A61K 2039/57

(57) ABSTRACT

Disclosed are synthetic nanocarrier compositions and immune checkpoint inhibitor compositions and related methods for administration to a subject.

### RELATED APPLICATIONS

This application claims the benefit of priority under 35 U.S.C. §119 to U.S. Provisional Application No. 62/017,101, filed Jun. 25, 2014, the entire contents of which are incorporated herein by reference.

### FIELD OF THE INVENTION

This invention relates to methods of administering a synthetic nanocarrier composition, that comprises an antigen and an immunostimulator, and an immune checkpoint inhibitor composition to a subject, and related compositions.

### SUMMARY OF THE INVENTION

In one aspect, a method comprising administering a synthetic nanocarrier composition, comprising a first population of synthetic nanocarriers that are attached to an antigen and a second population of synthetic nanocarriers that are attached to an immunostimulator, and an immune checkpoint inhibitor composition to a subject is provided.

In one embodiment of any one of the methods, compositions, or kits provided herein the first population of synthetic nanocarriers and the second population of synthetic nanocarriers are the same population of synthetic nanocarriers. In another embodiment of any one of the methods, compositions, or kits provided herein the first population of synthetic nanocarriers and the second population of synthetic nanocarriers are different populations of synthetic nanocarriers.

In one embodiment of any one of the methods provided herein, the method further comprises providing or obtaining the synthetic nanocarrier composition and providing or obtaining the immune checkpoint inhibitor composition. In another embodiment of any one of the methods provided herein, the antigen and immunostimulator are encapsulated within the synthetic nanocarriers of the synthetic nanocarrier composition. In another embodiment of any one of the methods provided herein, the synthetic nanocarrier composition and immune checkpoint inhibitor composition are administered concomitantly to the subject. In another embodiment of any one of the methods provided herein, the synthetic nanocarrier composition is administered prior to the immune checkpoint inhibitor composition. In another embodiment of any one of the methods provided herein, the synthetic nanocarrier composition is administered at least four times to the subject and the immune checkpoint inhibitor composition of administered at least three times to the subject. In another embodiment of any one of the methods provided herein, the synthetic nanocarrier composition and immune checkpoint inhibitor composition are each administered five times to the subject. In another embodiment of any one of the methods provided herein, the subject has or is at risk of having cancer or an infection or infectious disease. In another embodiment of any one of the methods provided herein, the subject has or is at risk of having a chronic infection.

In another embodiment of any one of the methods provided herein, the synthetic nanocarrier composition and immune checkpoint inhibitor composition are administered to a subject according to a protocol that has been shown to result in an enhanced immune response against the antigen. In another embodiment of any one of the methods provided herein, the synthetic nanocarrier composition and immune checkpoint inhibitor composition are administered to a subject according to a protocol that has been shown to result in a reduced immunosuppressive immune response against the antigen. In another embodiment of any one of the methods provided herein, the synthetic nanocarrier composition and immune checkpoint inhibitor composition are administered to a subject according to a protocol that has been shown to be effective in the treatment of any one of the diseases provided herein. In another embodiment of any one of the methods provided herein, the method further comprises determining the protocol.

In another embodiment of any one of the methods provided herein, the method further comprises assessing an immune response against the antigen in the subject prior to, during or subsequent to administration of the synthetic nanocarrier composition and/or immune checkpoint inhibitor composition.

In another embodiment of any one of the methods provided herein, the method further comprises administering at least one dose of the synthetic nanocarrier composition before the step of administering the synthetic nanocarrier composition and immune checkpoint inhibitor composition to the subject.

In another embodiment of any one of the methods provided herein, the administering is by intravenous, intraperitoneal or subcutaneous administration.

In another aspect, a composition or kit comprising a synthetic nanocarrier dose, wherein the synthetic nanocarrier dose comprises a first population of synthetic nanocarriers that comprise an antigen and second population of synthetic nanocarriers that comprise an immunostimulator, and a dose of an immune checkpoint inhibitor composition is provided. In one embodiment, the composition or kit is for use in any one of the methods provided herein. In another embodiment of any one of the compositions or kits, the composition or kit further comprises a pharmaceutically acceptable carrier.

In another embodiment of any one of the compositions or kits, the antigen and immunostimulator are encapsulated within the synthetic nanocarriers of the synthetic nanocarrier composition.

In another embodiment of any one of the compositions or kits, the synthetic nanocarrier dose and immune checkpoint inhibitor dose are contained in separate containers. In another embodiment of any one of the compositions or kits, the synthetic nanocarrier dose and immune checkpoint inhibitor dose are contained in the same container. In another embodiment of any one of the compositions or kits, the composition or kit further comprises instructions for use.

In one embodiment of any one of the methods, compositions or kits provided herein, the immunostimulator comprises a stimulator of a Toll-like receptor, RIG-1 or NOD-like receptor (NLR), mineral salt, MPL A of a bacterium, saponin, liposome, synthesized or specifically prepared microparticle or microcarrier such as a bacteria-derived outer membrane vesicle (OMV) of N. gonorrheae, Chlamydia trachomatis or other, chitosan particle, depot-forming agent, specifically modified or prepared peptide, bacterial toxoid or toxin fragment or immunostimulatory DNA or RNA.

In another embodiment of any one of the methods, compositions, or kits provided herein, the immune checkpoint inhibitor is an inhibitor of the PD-1/PD-L1, CTLA4/B7-1, TIM-3, LAG3, B7-He or H4 pathway. In another embodiment of any one of the methods, compositions, or kits provided herein, the immune checkpoint inhibitor is an antibody. In another embodiment of any one of the methods, compositions, or kits provided herein, the antibody is a monoclonal antibody. In another embodiment of any one of the methods, compositions, or kits provided herein, the antibody is an anti-PD-1 ligand antibody. In another embodiment of any one of the methods, compositions, or kits provided herein the antibody is 10F.9G2, BioXCell, Catalog # BE0101.

In another embodiment of any one of the methods, compositions, or kits provided herein, a load of the antigen and/or immunostimulator attached to the synthetic nanocarriers, on average across the population of synthetic nanocarriers, is between 0.1% and 50%. In another embodiment of any one of the methods, compositions, or kits provided herein, the load is between 0.1% and 20%. In another embodiment of any one of the methods, compositions, or kits provided the load is between 0.1% and 10%. In another embodiment of any one of the methods, compositions, or kits provided, the load of the antigen is between 0.75-2% and the load of the immunosuppressant is between 4-10%. In another embodiment of any one of the methods, compositions, or kits provided, the load of the antigen is between 0.75-1% and the load of the immunosuppressant is between 4-7%.

In another embodiment of any one of the methods, compositions, or kits provided herein, the synthetic nanocarriers comprise lipid nanoparticles, polymeric nanoparticles, metallic nanoparticles, surfactant-based emulsions, dendrimers, buckyballs, nanowires, virus-like particles or peptide or protein particles. In another embodiment of any one of the methods, compositions, or kits provided herein, the synthetic nanocarriers comprise lipid nanoparticles. In another embodiment of any one of the methods, compositions, or kits provided herein, the synthetic nanocarriers comprise liposomes. In another embodiment of any one of the methods, compositions, or kits provided herein, the synthetic nanocarriers comprise metallic nanoparticles. In another embodiment of any one of the methods, compositions, or kits provided herein, the metallic nanoparticles comprise gold nanoparticles.

In another embodiment of any one of the methods, compositions, or kits provided herein, the synthetic nanocarriers comprise polymeric nanoparticles. In another embodiment of any one of the methods, compositions, or kits provided herein, the polymeric nanoparticles comprise polymer that is a non-methoxy-terminated, pluronic polymer. In another embodiment of any one of the methods, compositions, or kits provided herein, the polymeric nanoparticles comprise a polyester, polyester attached to a polyether, polyamino acid, polycarbonate, polyacetal, polyketal, polysaccharide, polyethyloxazoline or polyethyleneimine. In another embodiment of any one of the methods, compositions, or kits provided herein, the polyester comprises a poly(lactic acid), poly(glycolic acid), poly(lactic-co-glycolic acid) or polycaprolactone. In another embodiment of any one of the methods, compositions, or kits provided herein, the polymeric nanoparticles comprise a polyester and a polyester attached to a polyether. In another embodiment of any one of the methods, compositions, or kits provided herein, the polyether comprises polyethylene glycol or polypropylene glycol.

In another embodiment of any one of the methods, compositions, or kits provided herein, the mean of a particle size distribution obtained using dynamic light scattering of the synthetic nanocarriers is a diameter greater than 100 nm. In another embodiment of any one of the methods, compositions, or kits provided herein, the diameter is greater than 150 nm. In another embodiment of any one of the methods, compositions, or kits provided herein, the diameter is greater than 200 nm. In another embodiment of any one of the methods, compositions, or kits provided herein, the diameter is greater than 250 nm. In another embodiment of any one of the methods, compositions, or kits provided herein, the diameter is greater than 300 nm. In another embodiment of any one of the methods, compositions, or kits provided herein, the diameter is less than 5 μm. In another embodiment of any one of the methods, compositions, or kits provided herein, the diameter is less than 4 μm. In another embodiment of any one of the methods, compositions, or kits provided herein, the diameter is less than 3 μm. In another embodiment of any one of the methods, compositions, or kits provided herein, the diameter is less than 2 μm. In another embodiment of any one of the methods, compositions, or kits provided herein, the diameter is less than 1 μm. In another embodiment of any one of the methods, compositions, or kits provided herein, the diameter is less than 500 nm. In another embodiment of any one of the methods, compositions, or kits provided herein, the diameter is less than 400 nm. In another embodiment of any one of the methods, compositions, or kits provided herein, the diameter is less than 350 nm. In another embodiment of any one of the methods, compositions, or kits provided herein, the diameter is less than 300 nm. In another embodiment of any one of the methods, compositions, or kits provided herein, the diameter is less than 250 nm. In another embodiment of any one of the methods, compositions, or kits provided herein, the mean of a particle size distribution obtained using dynamic light scattering of the synthetic nanocarriers is a diameter between 125-200 nm. In another embodiment of any one of the methods, compositions, or kits provided herein, the diameter is between 130-160 nm.

In another embodiment of any one of the methods, compositions, or kits provided herein, the diameter of at least 80% of the synthetic nanocarriers falls within 20% of the mean diameter. In another embodiment of any one of the methods, compositions, or kits provided herein, the diameter of at least 90% of the synthetic nanocarriers falls within 20% of the mean diameter. In another embodiment of any one of the methods, compositions, or kits provided herein, the diameter of at least 95% of the synthetic nanocarriers falls within 20% of the mean diameter. In another embodiment of any one of the methods, compositions, or kits provided herein, the diameter of the synthetic nanocarriers falls within 10% of the mean diameter. In another embodiment of any one of the methods, compositions, or kits provided herein, the diameter of the synthetic nanocarriers falls within 5% of the mean diameter.

In another embodiment of any one of the methods, compositions, or kits provided herein, an aspect ratio of the synthetic nanocarriers is greater than 1:1, 1:1.2, 1:1.5, 1:2, 1:3, 1:5, 1:7 or 1:10.

In another embodiment of any one of the methods, compositions, or kits provided herein, the synthetic nanocarrier composition targets antigen-presenting cells without comprising a specific targeting component. In another embodiment of any one of the methods, compositions, or kits provided herein the synthetic nanocarriers do not comprise a T cell costimulatory molecule on its surface. In another embodiment of any one of the methods, compositions, or kits provided herein the synthetic nanocarriers may comprise a T cell costimulatory molecule on its surface but also comprises the T cell costimulatory molecule within the synthetic nanocarriers. In another embodiment of any one of the methods, compositions, or kits provided herein the synthetic nanocarriers may comprise a T cell costimulatory molecule that is encapsulated within the synthetic nanocarriers. In another embodiment of any one of the methods, compositions, or kits provided herein the synthetic nanocarriers do not comprise a CD28 binding ligand.

In another aspect, a method comprising producing any one of the synthetic nanocarriers compositions provided herein and producing any one of the immune checkpoint inhibitor compositions provided herein is provided. In one embodiment of any one of the methods provided, the synthetic nanocarrier composition and immune checkpoint inhibitor composition are combined, such as in a kit.

In another embodiment of any one of the methods, compositions, or kits provided herein, the synthetic nanocarriers are solid synthetic nanocarriers. In another embodiment of any one of the methods, compositions, or kits provided herein, the synthetic nanocarriers do not comprise albumin nanoparticles. In another embodiment of any one of the methods, compositions, or kits provided herein, the synthetic nanocarriers do not comprise albumin. In another embodiment of any one of the methods, compositions, or kits provided herein, the synthetic nanocarriers are not lipid-based. In another embodiment of any one of the methods, compositions, or kits provided herein, the synthetic nanocarriers do not comprise lipids. In another embodiment of any one of the methods, compositions, or kits provided herein, the synthetic nanocarriers do not comprise liposomes.

In another embodiment of any one of the methods, compositions, or kits provided herein, the synthetic nanocarriers are designed to be phagocytosed and taken up, such as by antigen-presenting cells. In another embodiment of any one of the methods, compositions, or kits provided herein, the synthetic nanocarriers do not comprise a molecule that specifically targets a cell surface receptor. In another embodiment of any one of the methods, compositions, or kits provided herein, the synthetic nanocarriers do not comprise an antigen-presenting complex. In another embodiment of any one of the methods, compositions, or kits provided herein, the synthetic nanocarriers do not comprise a conjugated antigen-presenting complex. In another embodiment of any one of the methods, compositions, or kits provided herein, the synthetic nanocarriers do not comprise an unconjugated antigen-presenting complex.

In another embodiment of any one of the methods, compositions, or kits provided herein, the immune checkpoint inhibitor is soluble and not coupled to any synthetic nanocarriers. In another embodiment of any one of the methods, compositions, or kits provided herein, the immune checkpoint inhibitor is attached to a synthetic nanocarrier. In another embodiment of any one of the methods, compositions, or kits provided herein, the immune checkpoint inhibitor is attached to a different population of synthetic nanocarriers than the first or second population of synthetic nanocarriers. In another embodiment of any one of the methods, compositions, or kits provided herein, the immune checkpoint inhibitor is a PD-1/PD-1L inhibitor.

In another aspect, any one of the methods or compositions described herein, including any one of those in the Examples and Figures, are provided.

### BRIEF DESCRIPTION OF FIGURES

FIG. 1A shows the tumor burden following administration of an immunostimulation regimen. FIG. 1B shows the percent survival following administration of an immunostimulation regimen. FIG. 1C shows the tumor burden following administration of empty synthetic nanocarriers.

FIG. 2A shows the tumor burden following subcutaneous administration of synthetic nanocarriers carrying the TRP2 peptide epitope and the TLR7/8 agonist R848 on days 1, 4, 11, 18. FIG. 2B shows the tumor burden following subcutaneous administration of synthetic nanocarriers carrying the TRP2 peptide epitope and the TLR7/8 agonist R848 and an anti-PD-1 antibody after an initial nanocarrier treatment. FIG. 2C shows tumor burden following intraperitoneal administration of an anti-PD-1 antibody on days 2, 6, 9, without the synthetic nanocarriers. Individual animal tumor growth curves are shown.

FIGS. 3A-3C shows the systemic cytokine production in mice after nanocarrier inoculation. FIGS. 3A, 3B, and 3C show the production of TNF-α, IL-6, and IL-12 in experimental groups, respectively. Sera from groups of three mice were pooled and analyzed by ELISA.

FIGS. 4A and 4B show that TNF-α and IL-6 were induced in sera of NC-CpG- and free CpG-inoculated animals.

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to “a DNA molecule” includes a mixture of two or more such DNA molecules or a plurality of such DNA molecules, reference to “an immunostimulator” includes mixture of two or more such immunostimulator molecules or a plurality of such immunostimulator molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### A. INTRODUCTION

In the treatment of challenging diseases, such as cancer, generation of immune responses to tumor-related antigens is often difficult due to the complexity of the immune response and the presence of multiple refractory or immunosuppressive pathways that prevent generation of a robust immune response. Similarly, in the context of chronic infections and many infectious diseases, the persistent presence of antigens can induce immune exhaustion in the subject, rendering the subject unable to generate an immune response to the antigen and unable to develop immune effector cells de novo.

In addition, it has generally been thought that a proinflammatory response is needed for efficacious use of vaccines in combination with compounds like immune checkpoint inhibitors (See, for example, WO2014/144885). Synthetic nanocarriers, such as those described herein, however result in no or limited induction of systemic proinflammatory cytokines as demonstrated in Examples 10 and 11 below. This is further described in U.S. Publication No. 20120027806, the contents of which are incorporated herein by reference in their entirety. Surprisingly, however, the inventors have found that treatment with a synthetic nanocarrier vaccine comprising synthetic nanocarriers with an immunostimulator, even one that is encapsulated within the synthetic nanocarriers, in combination with an immune checkpoint inhibitor is not only efficacious but results in synergistic effects.

The inventors have unexpectedly and surprisingly discovered that the problems and limitations noted above can be overcome by practicing the invention disclosed herein. In particular, it has been unexpectedly and surprisingly discovered that administration of synthetic nanocarrier compositions comprising an antigen and an immunostimulator as well as an immune checkpoint inhibitor can result in a synergistic effect even without a significant proinflammatory response. Without being bound to a theory, it is thought that the synergistic effect is the result of blocking immunosuppressive pathways and/or the enhancement of lasting adaptive immune responses to antigen. Provided herein are methods and related compositions and kits for the administration of synthetic nanocarrier compositions, comprising an antigen and an immunostimulator, as well as an immune checkpoint inhibitor to a subject.

In embodiments of any one of the methods, compositions or kits provided, the immune responses that are generated are clinically effective. In some embodiments of any one of the methods provided, the subject to which the compositions are administered may have or be at risk of having cancer, an infection or infectious disease. In other embodiments of any one of the methods provided, the compositions are administered to a subject, such as a human, is according to a protocol that has been shown to result in an enhanced immune response against an antigen or result in a reduced immunosuppressive immune response to the antigen.

The invention will now be described in more detail below.

### B. DEFINITIONS

“Administering” or “administration” or “administer” means providing a material to a subject in a manner that is pharmacologically useful. The term includes causing to be administered. “Causing to be administered” means causing, urging, encouraging, aiding, inducing or directing, directly or indirectly, another party to administer the material.

“Amount effective” is any amount of a composition provided herein that produces one or more desired responses, such as one or more desired immune responses, including a reduced immunosuppressive immune response against an antigen. This amount can be for in vitro or in vivo purposes. For in vivo purposes, the amount can be one that a clinician would believe may have a clinical benefit for a subject in need of an immune response to an antigen. An effective amount that a clinician would believe may have a clinical benefit for such a subject is also referred to herein as a “clinically effective amount”. In embodiments, both the humoral immune response and the CTL immune response that is elicited by a composition provided herein results in a clinical effect from each of these arms of the immune system. In other embodiments, clinically effective amounts are effective amounts that can be helpful in the treatment of a subject with a disease or condition in which an immune response to an antigen would provide a benefit. Such subjects include, in some embodiments, those that have or are at risk of having cancer, an infection or infectious disease. Subjects also include those that have a chronic infection. Chronic infections are known to those of ordinary skill in the art (See, for example, JEM, Ha et al. 205 (3): 543-555, 2008) and include viral infections such as, but not limited to, HIV, hepatitis B virus (HBV), hepatitis C virus (HCV), and lymphoytic choriomeningitis virus (LCMV) infections. In other embodiments, subjects include those that have or are at risk of having a chronic infection such as any one of the foregoing or malaria, leischmaniasis, a human filovirus infection, a togavirus infection, a alphavirus infection, an arenavirus infection, a bunyavirus infection, a flavivirus infection, a human papillomavirus infection, or a human influenza A virus infection.

A subject's immune response can be monitored by routine methods. An amount that is effective to produce the desired immune responses as provided herein can also be an amount of a composition provided herein that produces a desired therapeutic endpoint or a desired therapeutic result. In another embodiment, the immunity persists in the subject. In still another embodiment, the immunity results or persists due to the administration of a composition provided herein according to a protocol as provided herein.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason.

“Antigen” means a B cell antigen or T cell antigen. “Type(s) of antigens” means molecules that share the same, or substantially the same, antigenic characteristics. In some embodiments, antigens may be proteins, polypeptides, peptides, lipoproteins, glycolipids, polynucleotides, polysaccharides or are contained or expressed in cells. In any one of the methods or compositions or kits provided herein, the antigen is a carbohydrate associated with an infectious agent. In any one of the methods or compositions or kits provided herein, the antigen is a glycoprotein or glycopeptide associated with an infectious agent. The infectious agent can be a bacterium, virus, fungus, protozoan, or parasite. In any one of the methods or compositions or kits provided herein, the antigen is associated with a tumor or a type of cancer.

“Antigens associated” with a disease, disorder or condition provided herein are antigens that can generate an immune response against, as a result of, or in conjunction with the disease, disorder or condition; that cause the disease, disorder or condition (or a symptom or effect thereof); and/or that is a marker of the disease, disorder or condition. In some embodiments, such as with cancer, such antigens are expressed in or on diseased cells, such as cancer or tumor cells, but not in or on normal or healthy cells (or non-diseased cells). Such antigens can also comprise an antigen that is expressed in or on diseased cells and on normal or healthy cells (or non-diseased cells) but is expressed in or on diseased cells at a greater level than on normal or healthy cells (or non-diseased cells). Preferably, the use of an antigen associated with a disease or condition provided herein will not lead to a substantial or detrimental immune response against normal or healthy cells or will lead to a beneficial immune response against the disease or condition that outweighs any immune response against normal or healthy cells (or non-diseased cells).

An “at risk” subject is one in which a health practitioner believes has a chance of having a disease, disorder or condition or is one a health practitioner believes would benefit from the compositions and methods provided. In an embodiment of any one of the methods, compositions or kits provided herein, the subject is one that is at risk of having cancer, infection, or infectious disease.

“Attach” or “Attached” or “Attaches” (and the like, such as “couple”) means to associate, such as chemically, one entity (for example a moiety) with another. In some embodiments, the attaching is covalent, meaning that the attaching occurs in the context of the presence of a covalent bond between the two entities. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of attaching. In some embodiments of any one of the methods, compositions or kits, the antigen and immunostimulator are attached to the synthetic nanocarriers.

“Concomitantly” means administering two or more materials/agents to a subject in a manner that is correlated in time, preferably sufficiently correlated in time so as to provide a modulation in a physiologic or immunologic response, and even more preferably the two or more materials/agents are administered in combination. In embodiments, concomitant administration may encompass administration of two or more compositions within a specified period of time, preferably within 1 month, more preferably within 1 week, still more preferably within 1 day, and even more preferably within 1 hour. In embodiments, the compositions may be repeatedly administered concomitantly, that is concomitant administration on more than one occasion, such as may be provided in the Examples.

“Determining” or “determine” means to ascertain a factual relationship. Determining may be accomplished in a number of ways, including but not limited to performing experiments, or making projections. For instance, a dose of an antigen, an immunostimulator, or an immune checkpoint inhibitor may be determined by starting with a test dose and using known scaling techniques (such as allometric or isometric scaling) to determine the dose for administration. Such may also be used to determine a protocol as provided herein. In another embodiment, the dose may be determined by testing various doses in a subject, i.e. through direct experimentation based on experience and guiding data. “Determining” or “determine” comprises “causing to be determined.” “Causing to be determined” means causing, urging, encouraging, aiding, inducing or directing or acting in coordination with an entity for the entity to ascertain a factual relationship; including directly or indirectly, or expressly or impliedly.

“Dosage form” means a pharmacologically and/or immunologically active material in a medium, carrier, vehicle, or device suitable for administration to a subject. Any one of the compositions or doses provided herein may be in a dosage form.

“Dose” refers to a specific quantity of a pharmacologically and/or immunologically active material for administration to a subject for a given time.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier.

“Generating” means causing an action, such as an immune response against an antigen to occur, either directly oneself or indirectly, such as, but not limited to, an unrelated third party that takes an action through reliance on one's words or deeds.

“Identifying a subject” is any action or set of actions that allows a clinician to recognize a subject as one who may benefit from the methods and compositions provided herein. Preferably, the identified subject is one who is in need of an immune response, or a change in an immune response, to an antigen. Such subjects include any subject that has or is at risk of having any of the disease or conditions provided herein. The action or set of actions may be either directly oneself or indirectly, such as, but not limited to, an unrelated third party that takes an action through reliance on one's words or deeds.

“Identifying” is any action or set of actions that allows a clinician to recognize a subject as one who may benefit from the methods and compositions provided herein. Such subjects include any subject that has or is at risk of having any of the disease or conditions provided herein. The action or set of actions may be either directly oneself or indirectly, such as, but not limited to, an unrelated third party that takes an action through reliance on one's words or deeds.

An “immune checkpoint inhibitor” is any molecule that directly or indirectly inhibits, partially or completely, an immune checkpoint pathway. Without wishing to be bound by any particular theory, it is generally thought that immune checkpoint pathways function to turn on or off aspects of the immune system, particularly T cells. Following activation of a T cell, a number of inhibitory receptors can be upregulated and present on the surface of the T cell in order to suppress the immune response at the appropriate time. In the case of persistent immune stimulation, such as with chronic viral infection, for example, immune checkpoint pathways can suppress the immune response and lead to immune exhaustion. Aspects of the disclosure are related to the observation that inhibiting such immune checkpoint pathways and administering synthetic nanocarrier compositions comprising antigens and immunostimulators, can result in the generation of enhanced immune responses to the antigen and/or a reduction in immunosuppressive immune responses against the antigen. Examples of immune checkpoint pathways include, without limitation, PD-1/PD-L1, CTLA4/B7-1, TIM-3, LAG3, By-He, H4, HAVCR2, ID01, CD276 and VTCN1. In the instance of the PD-1/PD-L1 immune checkpoint pathway, an inhibitor may bind to PD-1 or to PD-L1 and prevent interaction between the receptor and ligand. Therefore, the inhibitor may be an anti-PD-1 antibody or anti-PD-L1 antibody. Similarly, in the instance of the CTLA4/B7-1 immune checkpoint pathway, an inhibitor may bind to CTLA4 or to B7-1 and prevent interaction between the receptor and ligand. Further examples of immune checkpoint inhibitors can be found, for example, in WO2014/144885. Such immune checkpoint inhibitors are incorporated by reference herein. In some embodiments of any one of the methods, compositions or kits provided, the immune checkpoint inhibitor is a small molecule inhibitor of an immune checkpoint pathway. In some embodiments of any one of the methods, compositions or kits provided, the immune checkpoint inhibitor is a polypeptide that inhibits an immune checkpoint pathway. In some embodiments of any one of the methods, compositions or kits provided, the inhibitor is a fusion protein. In some embodiments of any one of the methods, compositions or kits provided, the immune checkpoint inhibitor is an antibody. In some embodiments of any one of the methods, compositions or kits provided, the antibody is a monoclonal antibody. Non-limiting examples of immune checkpoint inhibitors include fully human monoclonal antibodies, such as BMS-936558/MDX-1106, BMS-936559/MDX-1105, ipilimumab/Yervoy, and tremelimumab; humanized antibodies, such as CT-011 and MK-3475; and fusion proteins, such as AMP-224.

“Immunostimulator” as used herein refers to a compound that can stimulate an immune response in a subject, and may include an adjuvant. An immunostimulator is an agent that does not constitute a specific antigen, but, in some embodiments, can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (ASO4), MPL A of above-mentioned bacteria separately, saponins, such as QS-21, Quil-A, ISCOMs, ISCOMATRIX™, emulsions such as MF59™, Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments.

In embodiments, immunostimulators comprise agonists for pattern recognition receptors (PRR), including, but not limited to Toll-Like Receptors (TLRs), specifically TLRs 2, 3, 4, 5, 7, 8, 9 and/or combinations thereof. In other embodiments, immunostimulators comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381 (Sumitomo Pharmaceutical Company), U.S. Published Patent Application 2010/0075995 to Biggadike et al., or WO 2010/018132 to Campos et al.; immunostimulatory DNA; or immunostimulatory RNA. In specific embodiments, synthetic nanocarriers incorporate as immunostimulators compounds that are agonists for toll-like receptors (TLRs) 7 & 8 (“TLR 7/8 agonists”). Of utility are the TLR 7/8 agonist compounds disclosed in U.S. Pat. No. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred immunostimulators comprise imiquimod and resiquimod (also known as R848). In specific embodiments, an immunostimulator may be an agonist for the DC surface molecule CD40. In certain embodiments, to stimulate immunity rather than tolerance, a synthetic nanocarrier incorporates an immunostimulator that promotes DC maturation (needed for priming of naive T cells) and the production of cytokines, such as type I interferons, which promote antibody immune responses. In embodiments, immunostimulators also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an immunostimulator may be a TLR-4 agonist, such as bacterial lipopolysaccharide (LPS), VSV-G, and/or HMGB-1. In some embodiments, immunostimulators may comprise TLR-5 agonists, such as flagellin, or portions or derivatives thereof, including but not limited to those disclosed in U.S. Pat. Nos. 6,130,082, 6,585,980, and 7,192,725. In specific embodiments, synthetic nanocarriers incorporate a ligand for Toll-like receptor (TLR)-9, such as immunostimulatory DNA molecules comprising CpGs, which induce type I interferon secretion, and stimulate T and B cell activation leading to increased antibody production and cytotoxic T cell responses (Krieg et al., CpG motifs in bacterial DNA trigger direct B cell activation. Nature. 1995. 374:546-549; Chu et al. CpG oligodeoxynucleotides act as adjuvants that switch on T helper 1 (Th1) immunity. J. Exp. Med. 1997. 186:1623-1631; Lipford et al. CpG-containing synthetic oligonucleotides promote B and cytotoxic T cell responses to protein antigen: a new class of vaccine adjuvants. Eur. J. Immunol. 1997. 27:2340-2344; Roman et al. Immunostimulatory DNA sequences function as T helper-1-promoting adjuvants. Nat. Med. 1997. 3:849-854; Davis et al. CpG DNA is a potent enhancer of specific immunity in mice immunized with recombinant hepatitis B surface antigen. J. Immunol. 1998. 160:870-876; Lipford et al., Bacterial DNA as immune cell activator. Trends Microbiol. 1998. 6:496-500; U.S. Pat. No. 6,207,646 to Krieg et al.; U.S. Pat. No. 7,223,398 to Tuck et al.; U.S. Pat. No. 7,250,403 to Van Nest et al.; or U.S. Pat. No. 7,566,703 to Krieg et al.

In some embodiments, immunostimulators may be proinflammatory stimuli released from necrotic cells (e.g., urate crystals). In some embodiments, immunostimulators may be activated components of the complement cascade (e.g., CD21, CD35, etc.). In some embodiments, immunostimulators may be activated components of immune complexes. The immunostimulators also include complement receptor agonists, such as a molecule that binds to CD21 or CD35. In some embodiments, the complement receptor agonist induces endogenous complement opsonization of the synthetic nanocarrier. In some embodiments, immunostimulators are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

An “infection” or “infectious disease” is any condition or disease caused by a microorganism, pathogen or other agent, such as a bacterium, fungus, prion or virus.

“Load” is the amount of the a component attached to a synthetic nanocarrier based on the total weight (such as the dry weight) of materials in an entire synthetic nanocarrier (weight/weight). Generally, the load is calculated as an average across a population of synthetic nanocarriers. In embodiments of any one of the compositions and methods provided, the load can be calculated as follows: Approximately 3 mg of synthetic nanocarriers are collected and centrifuged to separate supernatant from synthetic nanocarrier pellet. Acetonitrile is added to the pellet, and the sample is sonicated and centrifuged to remove any insoluble material. The supernatant and pellet are injected on RP-HPLC and absorbance is read at 278 nm. The μg found in the pellet is used to calculate % entrapped (load), μg in supernatant and pellet are used to calculate total μg recovered.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of inventive synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., diameter) may be obtained by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicies of the sample. The effective diameter, or mean of the distribution, can then reported. “Dimension” or “size” or “diameter” of synthetic nanocarriers means the mean of a particle size distribution obtained using dynamic light scattering in some embodiments.

“Pharmaceutically acceptable excipient” or “pharmaceutically acceptable carrier” means a pharmacologically inactive material used together with an active material to formulate the compositions. Pharmaceutically acceptable excipients or carriers comprise a variety of materials known in the art, including but not limited to saccharides (such as glucose, lactose, and the like), preservatives such as antimicrobial agents, reconstitution aids, colorants, saline (such as phosphate buffered saline), and buffers.

“Protocol” refers to any dosing regimen of one or more substances to a subject. A dosing regimen may include the amount, frequency, rate, duration and/or mode of administration. In some embodiments, such a protocol may be used to administer one or more compositions of the invention to one or more test subjects. Immune responses in these test subjects can then be assessed to determine whether or not the protocol was effective in generating a desired immune response, such as an enhanced immune response against the antigen, or reducing an immune response, such as an immunosuppressive immune response against the antigen. Any other therapeutic and/or prophylactic effects may also be assessed instead of or in addition to the aforementioned immune responses. Whether or not a protocol had a desired effect can be determined using any of the methods provided herein or otherwise known in the art. For example, a population of cells may be obtained from a subject to which a composition provided herein has been administered according to a specific protocol in order to determine whether or not specific immune cells, cytokines, antibodies, etc. were generated, activated, etc. Useful methods for detecting the presence and/or number of immune cells include, but are not limited to, flow cytometric methods (e.g., FACS) and immunohistochemistry methods. Antibodies and other binding agents for specific staining of immune cell markers, are commercially available. Such kits typically include staining reagents for multiple antigens that allow for FACS-based detection, separation and/or quantitation of a desired cell population from a heterogeneous population of cells.

“Providing” means an action or set of actions that an individual performs that supply a needed item or set of items or method for practicing of the present invention. The action or set of actions may be taken either directly oneself or indirectly.

“Providing a subject” is any action or set of actions that causes a clinician to come in contact with a subject and administer a composition provided herein thereto or to perform a method provided herein thereupon. The action or set of actions may be taken either directly oneself or indirectly. In an embodiment of any one of the methods provided herein, the method further comprises providing a subject.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, synthetic nanocarriers do not comprise chitosan. In certain other embodiments, the synthetic nanocarriers do not comprise chitosan. In other embodiments, inventive synthetic nanocarriers are not lipid-based nanoparticles. In further embodiments, inventive synthetic nanocarriers do not comprise a phospholipid.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., (3) the lithographically constructed nanoparticles of Published US Patent Application 20090028910 to DeSimone et al., (4) the disclosure of WO 2009/051837 to von Andrian et al., (5) the nanoparticles disclosed in Published US Patent Application 2008/0145441 to Penades et al., (6) the protein nanoparticles disclosed in Published US Patent Application 20090226525 to de los Rios et al., (7) the virus-like particles disclosed in published US Patent Application 20060222652 to Sebbel et al., (8) the nucleic acid coupled virus-like particles disclosed in published US Patent Application 20060251677 to Bachmann et al., (9) the virus-like particles disclosed in W02010047839A1 or W02009106999A2, (10) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010), (11) apoptotic cells, apoptotic bodies or the synthetic or semisynthetic mimics disclosed in U.S. Publication 2002/0086049, or (12) those of Look et al., Nanogel-based delivery of mycophenolic acid ameliorates systemic lupus erythematosus in mice” J. Clinical Investigation 123(4):1741-1749(2013). In embodiments, synthetic nanocarriers may possess an aspect ratio greater than 1:1, 1:1.2, 1:1.5, 1:2, 1:3, 1:5, 1:7, or greater than 1:10.

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, when synthetic nanocarriers comprise virus-like particles, the virus-like particles comprise non-natural immunostimulator (meaning that the VLPs comprise an immunostimulators other than naturally occurring RNA generated during the production of the VLPs). In embodiments, synthetic nanocarriers may possess an aspect ratio greater than 1:1, 1:1.2, 1:1.5, 1:2, 1:3, 1:5, 1:7, or greater than 1:10.

### C. COMPOSITIONS FOR USE IN THE INVENTIVE METHODS

Provided herein are methods and related compositions and kits for effective stimulation or reduction in immune responses in a subject. It has been found that synthetic nanocarriers that comprise an antigen and an immunostimulator that are attached can be administered with an immune checkpoint inhibitor to generate effective and immune responses to the antigen even if the synthetic nanocarriers do not result in a prosinflammatory response. The compositions and kits provided can be used for administration to a subject that has or is at risk of having cancer, an infection, or infectious disease.

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In an embodiment of any one of the methods or compositions provided herein, it is desirable to use a population of synthetic nanocarriers that is relatively uniform in terms of size, shape, and/or composition so that each synthetic nanocarrier has similar properties. As an example of any one of these embodiments, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers. The average diameter or dimension may be any one of the diameters or dimensions provided herein.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In some embodiments, synthetic nanocarriers can comprise one or more polymers. In some embodiments, such a polymer can be surrounded by a coating layer (e.g., liposome, lipid monolayer, micelle, etc.). In some embodiments, various elements (i.e., components) of the synthetic nanocarriers can be coupled with the polymer.

In some embodiments, a component can be covalently associated with a polymeric matrix. In some embodiments, covalent association is mediated by a linker. In some embodiments, a component can be noncovalently associated with a polymeric matrix. For example, in some embodiments, a component can be encapsulated within, surrounded by, and/or dispersed throughout a polymeric matrix. Alternatively or additionally, a component can be associated with a polymeric matrix by hydrophobic interactions, charge interactions, van der Waals forces, etc.

A wide variety of polymers and methods for forming polymeric matrices therefrom are known conventionally. In general, a polymeric matrix comprises one or more polymers.

The synthetic nanocarriers provided herein may be polymeric nanocarriers. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments, the synthetic nanocarriers comprise one or more polymers that comprise a polyester, polycarbonate, polyamide, or polyether, or unit thereof. In other embodiments, the polymer comprises poly(ethylene glycol) (PEG), poly(lactic acid), poly(glycolic acid), poly(lactic-co-glycolic acid), or a polycaprolactone, or unit thereof. In some embodiments, it is preferred that the polymer is biodegradable. Therefore, in these embodiments, it is preferred that if the polymer comprises a polyether, such as poly(ethylene glycol) or unit thereof, the polymer comprises a block-co-polymer of a polyether and a biodegradable polymer such that the polymer is biodegradable. In other embodiments, the polymer does not solely comprise a polyether or unit thereof, such as poly(ethylene glycol) or unit thereof. The one or more polymers may be comprised within a polymeric synthetic nanocarrier or may be comprised in a number of other different types of synthetic nanocarriers.

Examples of polymers suitable for use in the present invention also include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g. coupled) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines. In embodiments, the synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol) 5000-phosphatidylethanolamine; poly(ethylene glycol) 400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

Compositions for use in the methods according to the invention can comprise synthetic nanocarriers in combination with pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, synthetic nanocarriers are suspended in sterile saline solution for injection together with a preservative.

In embodiments, when preparing synthetic nanocarriers as carriers, methods for coupling the components to the synthetic nanocarriers may be useful. If the component is a small molecule it may be of advantage to attach the component to a polymer prior to the assembly of the synthetic nanocarriers. In embodiments, it may also be an advantage to prepare the synthetic nanocarriers with surface groups that are used to couple the component to the synthetic nanocarrier through the use of these surface groups rather than attaching the component to a polymer and then using this polymer conjugate in the construction of synthetic nanocarriers.

In certain embodiments, the coupling can be a covalent linker. In embodiments, components according to the invention can be covalently attached to the external surface via a 1,2,3-triazole linker formed by the 1,3-dipolar cycloaddition reaction of azido groups on the surface of the nanocarrier with the component containing an alkyne group or by the 1,3-dipolar cycloaddition reaction of alkynes on the surface of the nanocarrier with components containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, the covalent attaching may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component with the carboxylic acid group of a second component such as the nanocarrier. The amide bond in the linker can be made using any of the conventional amide bond forming reactions with suitably protected amino acids or antigens or immunostimulators and activated carboxylic acid such N-hydroxysuccinimide-activated ester.

A disulfide linker is made via the formation of a disulfide (S—S) bond between two sulfur atoms of the form, for instance, of R1-S—S—R2. A disulfide bond can be formed by thiol exchange of an antigen or immunostimulatorcontaining thiol/mercaptan group (—SH) with another activated thiol group on a polymer or nanocarrier or a nanocarrier containing thiol/mercaptan groups with a component containing activated thiol group.

A triazole linker, specifically a 1,2,3-triazole of the form

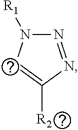

wherein R1 and R2 may be any chemical entities, is made by the 1,3-dipolar cycloaddition reaction of an azide attached to a first component such as the nanocarrier with a terminal alkyne attached to a second component. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The component is prepared with the presence of either an alkyne (if the polymer contains an azide) or an azide (if the polymer contains an alkyne) group. The component is then allowed to react with the nanocarrier via the 1,3-dipolar cycloaddition reaction with or without a catalyst which covalently attaches the component to the particle through the 1,4-disubstituted 1,2,3-triazole linker.

A thioether linker is made by the formation of a sulfur-carbon (thioether) bond in the form, for instance, of R1-S—R2. Thioether can be made by either alkylation of a thiol/mercaptan (—SH) group on one component with an alkylating group such as halide or epoxide on a second component such as the nanocarrier. Thioether linkers can also be formed by Michael addition of a thiol/mercaptan group on one component to an electron-deficient alkene group on a second component such as a polymer containing a maleimide group or vinyl sulfone group as the Michael acceptor. In another way, thioether linkers can be prepared by the radical thiol-ene reaction of a thiol/mercaptan group on one component with an alkene group on a second component such as a polymer or nanocarrier.

A hydrazone linker is made by the reaction of a hydrazide group on one component with an aldehyde/ketone group on the second component such as the nanocarrier.

A hydrazide linker is formed by the reaction of a hydrazine group on one component with a carboxylic acid group on the second component such as the nanocarrier. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component with an aldehyde or ketone group on the second component such as the nanocarrier.

An urea or thiourea linker is prepared by the reaction of an amine group on one component with an isocyanate or thioisocyanate group on the second component such as the nanocarrier.

An amidine linker is prepared by the reaction of an amine group on one component with an imidoester group on the second component such as the nanocarrier.

An amine linker is made by the alkylation reaction of an amine group on one component with an alkylating group such as halide, epoxide, or sulfonate ester group on the second component such as the nanocarrier. Alternatively, an amine linker can also be made by reductive amination of an amine group on one component with an aldehyde or ketone group on the second component such as the nanocarrier with a suitable reducing reagent such as sodium cyanoborohydride or sodium triacetoxyborohydride.

A sulfonamide linker is made by the reaction of an amine group on one component with a sulfonyl halide (such as sulfonyl chloride) group on the second component such as the nanocarrier.

A sulfone linker is made by Michael addition of a nucleophile to a vinyl sulfone. Either the vinyl sulfone or the nucleophile may be on the surface of the nanocarrier or attached to a component.

The component can also be conjugated to the nanocarrier via non-covalent conjugation methods. For examples, a negative charged component can be conjugated to a positive charged nanocarrier through electrostatic adsorption. A component containing a metal ligand can also be conjugated to a nanocarrier containing a metal complex via a metal-ligand complex.

In embodiments, the component can be attached to a polymer, for example polylactic acid-block-polyethylene glycol, prior to the assembly of the synthetic nanocarrier or the synthetic nanocarrier can be formed with reactive or activatible groups on its surface. In the latter case, the component may be prepared with a group which is compatible with the attachment chemistry that is presented by the synthetic nanocarriers' surface. In other embodiments, a component can be attached to VLPs or liposomes using a suitable linker. A linker is a compound or reagent that capable of coupling two molecules together. In an embodiment, the linker can be a homobifuntional or heterobifunctional reagent as described in Hermanson 2008. For example, an VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a component containing an acid group via the other end of the ADH linker on NC to produce the corresponding VLP or liposome peptide conjugate. For detailed descriptions of available conjugation methods, see Hermanson G T “Bioconjugate Techniques”, 2nd Edition Published by Academic Press, Inc., 2008.

In some embodiments, a component, such as an antigen or immunostimulator, may be isolated. Isolated refers to the element being separated from its native environment and present in sufficient quantities to permit its identification or use. This means, for example, the element may be (i) selectively produced by expression cloning or (ii) purified as by chromatography or electrophoresis. Isolated elements may be, but need not be, substantially pure. Because an isolated element may be admixed with a pharmaceutically acceptable excipient in a pharmaceutical preparation, the element may comprise only a small percentage by weight of the preparation. The element is nonetheless isolated in that it has been separated from the substances with which it may be associated in living systems, i.e., isolated from other lipids or proteins. Any of the elements provided herein may be isolated. Any of the antigens provided herein can be included in the compositions in isolated form.

### D. METHODS OF USING AND MAKING SYNTHETIC NANOCARRIER COMPOSITIONS

Synthetic nanocarriers may be prepared using a wide variety of methods known in the art. For example, synthetic nanocarriers can be formed by methods as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; US Patents 5578325 and 6007845; P. Paolicelli et al., “Surface-modified PLGA-based Nanoparticles that can Efficiently Associate and Deliver Virus-like Particles” Nanomedicine. 5(6):843-853 (2010)).

Various materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. No. 6,632,671 to Unger Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be coupled to the synthetic nanocarriers and/or the composition of the polymer matrix.

If particles prepared by any of the above methods have a size range outside of the desired range, particles can be sized, for example, using a sieve.

Elements of the synthetic nanocarriers may be attached to the overall synthetic nanocarrier, e.g., by one or more covalent bonds, or may be attached by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Alternatively or additionally, synthetic nanocarriers can be attached to elements directly or indirectly via non-covalent interactions. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such attachings may be arranged to be on an external surface or an internal surface of an synthetic nanocarrier. In embodiments, encapsulation and/or absorption is a form of attaching.

In embodiments, the synthetic nanocarriers can be attached to immunostimulators by any of the methods described herein. Such immunostimulators may include, but are not limited to stimulators of a Toll-like receptor (TLR), RIG-1 or NOD-like receptor (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (ASO4), MPL A of above-mentioned bacteria separately, saponins, such as QS-21,Quil-A, ISCOMs, ISCOMATRIX™, emulsions such as MF59™, Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, proteins, such as bacterial toxoids or toxin fragments; or immunostimulatory DNA or RNA. The doses of such other immunostimulators can be determined using conventional dose ranging studies.

Typical compositions that comprise synthetic nanocarriers may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention can comprise synthetic nanocarriers in combination with pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, synthetic nanocarriers are suspended in sterile saline solution for injection together with a preservative.

It is to be understood that the compositions can be made in any suitable manner, and the invention is in no way limited to the use of compositions that can be produced using the methods described herein. Selection of an appropriate method may require attention to the properties of the particular elements being associated.

In some embodiments, synthetic nanocarriers are manufactured under sterile conditions or are terminally sterilized. This can ensure that resulting composition are sterile and non-infectious, thus improving safety when compared to non-sterile compositions. This provides a valuable safety measure, especially when subjects receiving synthetic nanocarriers have immune defects, are suffering from infection, and/or are susceptible to infection. In some embodiments, synthetic nanocarriers may be lyophilized and stored in suspension or as lyophilized powder depending on the formulation strategy for extended periods without losing activity.

The compositions of the invention can be administered by a variety of routes, including or not limited to subcutaneous, intraperitoneal, etc. or by a combination of these routes.

Doses of dosage forms contain varying amounts of populations of synthetic nanocarriers or varying amounts of the immune checkpoint inhibitors, according to the invention. The amount of synthetic nanocarriers or inhibitors present in the dosage forms can be varied according to the nature of the elements present, the therapeutic benefit to be accomplished, and other such parameters. In embodiments, dose ranging studies can be conducted to establish optimal therapeutic amounts to be present in the dosage form. In embodiments, the synthetic nanocarriers and immune checkpoint inhibitors are present in the dosage form in an amount effective to generate an immune response to the antigen upon administration to a subject or a reduced immunosuppressive immune response to the antigen. It may be possible to determine amounts effective using conventional dose ranging studies and techniques in subjects. Dosage forms may be administered at a variety of frequencies. In a preferred embodiment, at least one administration of the dosage form is sufficient to generate a pharmacologically relevant response. In more preferred embodiment, at least two administrations, at least three administrations, or at least four administrations of the dosage form are utilized to ensure an effective response. Any one of the methods provided herein can include at least four or at least five administrations of any one of the synthetic nanocarrier compositions as provided herein and/or at least three or at least five administrations of immune checkpoint inhibitor compositions as provided herein.

The compositions and methods described herein can be used to induce, enhance, modulate, direct, or redirect an immune response. The compositions and methods described herein can be used for subject having or at risk of having conditions such as cancers, infections or infectious diseases, etc.

Examples of infectious disease include, but are not limited to, viral infectious diseases, such as AIDS, Chickenpox (Varicella), Common cold, Cytomegalovirus Infection, Colorado tick fever, Dengue fever, Ebola hemorrhagic fever, Hand, foot and mouth disease, Hepatitis, Herpes simplex, Herpes zoster, HPV, Influenza (Flu), Lassa fever, Measles, Marburg hemorrhagic fever, Infectious mononucleosis, Mumps, Norovirus, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease and Yellow fever; bacterial infectious diseases, such as Anthrax, Bacterial Meningitis, Botulism, Brucellosis, Campylobacteriosis, Cat Scratch Disease, Cholera, Diphtheria, Epidemic Typhus, Gonorrhea, Impetigo, Legionellosis, Leprosy (Hansen's Disease), Leptospirosis, Listeriosis, Lyme disease, Melioidosis, Rheumatic Fever, MRSA infection, Nocardiosis, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Typhus and Urinary Tract Infections; parasitic infectious diseases, such as African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, Trichomoniasis and Trypanosomiasis; fungal infectious disease, such as Aspergillosis, Blastomycosis, Candidiasis, Coccidioidomycosis, Cryptococcosis, Histoplasmosis, Tinea pedis (Athlete's Foot) and Tinea cruris; prion infectious diseases, such as Alpers' disease, Fatal Familial Insomnia, Gerstmann-Sträussler-Scheinker syndrome, Kuru and Variant Creutzfeldt-Jakob disease.

Examples of cancers include, but are not limited to breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia, e.g., B Cell CLL; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

The antigens for attaching to the synthetic nanocarriers can be antigens associated with any one of the diseases or conditions provided herein. These include antigens associated with cancers, infections or infectious diseases. Antigens associated with HIV, malaria, leischmaniasis, a human filovirus infection, a togavirus infection, a alphavirus infection, an arenavirus infection, a bunyavirus infection, a flavivirus infection, a human papillomavirus infection, a human influenza A virus infection, a hepatitis B infection or a hepatitis C infection are also included.

Examples of cancer antigens include E7 peptides, peptides from tyrosinase-related protein 2 (TRP2), HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, PR4D2, and the like. Further examples include MAGE, MART-1/Melan-A, gp100, Dipeptidyl peptidase IV (DPPIV), adenosine deaminase-binding protein (ADAbp), FAP, cyclophilin b, Colorectal associated antigen (CRC)--0017-1A/GA733, Carcinoembryonic Antigen (CEA) and its immunogenic epitopes CAP-1 and CAP-2, etv6, amll, prostatic acid phosphatase (PAP), Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-I or MAGE-II families) (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100Pme1117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20 and c-erbB-2.

In another embodiment, antigens associated with infection or infectious disease are associated with any of the infectious agents provided herein. In one embodiment, the infectious agent is a virus of the Adenoviridae, Picornaviridae, Herpesviridae, Hepadnaviridae, Flaviviridae, Retroviridae, Orthomyxoviridae, Paramyxoviridae, Papillomaviridae, Rhabdoviridae, Togaviridae or Paroviridae family. In still another embodiment, the infectious agent is adenovirus, coxsackievirus, hepatitis A virus, poliovirus, Rhinovirus, Herpes simplex virus, Varicella-zoster virus, Epstein-barr virus, Human cytomegalovirus, Human herpesvirus, Hepatitis B virus, Hepatitis C virus, yellow fever virus, dengue virus, West Nile virus, HIV, Influenza virus, Measles virus, Mumps virus, Parainfluenza virus, Respiratory syncytial virus, Human metapneumovirus, Human papillomavirus, Rabies virus, Rubella virus, Human bocarivus or Parvovirus B19. In yet another embodiment, the infectious agent is a bacteria of the Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema Vibrio or Yersinia genus. In a further embodiment, the infectious agent is Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycobacterium ulcerans, Mycoplasma pneumoniae, Neisseria gonorrhoeae, Neisseria meningitides, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae or Yersinia pestis. In another embodiment, the infectious agent is a fungus of the Candida, Aspergillus, Cryptococcus, Histoplasma, Pneumocystis or Stachybotrys genus. In still another embodiment, the infectious agent is C. albicans, Aspergillus fumigatus, Aspergillus flavus, Cryptococcus neoformans, Cryptococcus laurentii, Cryptococcus albidus, Cryptococcus gattii, Histoplasma capsulatum, Pneumocystis jirovecii or Stachybotrys chartarum.

In yet another embodiment, the antigen associated with infection or infectious disease is one that comprises VI, VII, E1A, E3-19K, 52K, VP1, surface antigen, 3A protein, capsid protein, nucleocapsid, surface projection, transmembrane proteins, UL6, UL18, UL35, UL38, UL19, early antigen, capsid antigen, Pp65, gB, p52, latent nuclear antigen-1, NS3, envelope protein, envelope protein E2 domain, gp120, p24, lipopeptides Gag (17-35), Gag (253-284), Nef (66-97), Nef (116-145), Pol (325-355), neuraminidase, nucleocapsid protein, matrix protein, phosphoprotein, fusion protein, hemagglutinin, hemagglutinin-neuraminidase, glycoprotein, E6, E7, envelope lipoprotein or non-structural protein (NS). In another embodiment, the antigen comprises pertussis toxin (PT), filamentous hemagglutinin (FHA), pertactin (PRN), fimbriae (FIM 2/3), V1sE; DbpA, OspA, Hia, PrpA, M1tA, L7/L12, D15, 0187, VirJ, Mdh, AfuA, L7/L12, out membrane protein, LPS, antigen type A, antigen type B, antigen type C, antigen type D, antigen type E, F1iC, FliD, Cwp84, alpha-toxin, theta-toxin, fructose 1,6-biphosphate-aldolase (FBA), glyceraldehydes-3-phosphate dehydrogenase (GPD), pyruvate:ferredoxin oxidoreductase (PFOR), elongation factor-G (EF-G), hypothetical protein (HP), T toxin, Toxoid antigen, capsular polysaccharide, Protein D, Mip, nucleoprotein (NP), RD1, PE35, PPE68, EsxA, EsxB, RD9, EsxV, Hsp70, lipopolysaccharide, surface antigen, Sp1, Sp2, Sp3, Glycerophosphodiester Phosphodiesterase, outer membrane protein, chaperone-usher protein, capsular protein (F1) or V protein. In yet another embodiment, the antigen is one that comprises capsular glycoprotein, Yps3P, Hsp60, Major surface protein, MsgC1, MsgC3, MsgC8, MsgC9 or SchS34.

Another aspect of the disclosure relates to kits. In some embodiments, the kit comprises a synthetic nanocarrier composition comprising a population of synthetic nanocarriers comprising an antigen and an immunostimulator and an immune checkpoint inhibitor composition. In such embodiments, the kit may also comprise a pharmaceutically acceptable carrier. The synthetic nanocarrier composition and immune checkpoint inhibitor composition can be contained within separate containers or within the same container in the kit. In some embodiments, the container is a vial or an ampoule. In some embodiments, the synthetic nanocarrier composition and/or immune checkpoint inhibitor composition are contained within a solution separate from the container, such that the synthetic nanocarrier composition and/or immune checkpoint inhibitor composition may be added to the container at a subsequent time. In some embodiments, the synthetic nanocarrier composition and/or immune checkpoint inhibitor composition are in lyophilized form in a separate container, such that they may be reconstituted at a subsequent time. In some embodiments, the kit further comprises instructions for reconstitution, mixing, administration, etc. In some embodiments, the instructions include a description of the methods described herein. Instructions can be in any suitable form, e.g., as a printed insert or a label. In some embodiments, the kit further comprises one or more syringes.

### EXAMPLES

### Example 1

### Synthetic Nanocarrier Formulation

### Materials

Synthetic oligonucleotide M362 CpG (5′-tcgtcgtcgttc:gaacgacgttgat-3′), (M362), was purchased from Oligo Factory (120 Jeffrey Ave, Holliston, Mass. 01746), custom manufacture number M362/Selecta Biosciences Lot Number: 1557, Poly(lactide-co-glycolide) polymer, (PLGA), with 54% lactide and 46% glycolide content and an inherent viscosity of 0.24 dL/g was purchased from Lakeshore Biomaterials (756 Tom Martin Drive, Birmingham, Ala. 35211), product code 5050 DLG 2.5 A. Poly(lactide-co-glycolide)-bl-poly(ethylene glycol) block co-polymer with a lactide to glycolide ratio of 75: 25, and a PEG block of approximately 14% by weight and Mw of 88,000 Da, inherent viscosity of 0.70 dL/g (PLGA-PEG), was purchased from Lakeshore Biomaterials (756 Tom Martin Drive, Birmingham, Ala. 35211), product code 7525 DLG PEG 2000 7E-P. EMPROVE® Polyvinyl Alcohol 4-88, USP, 85-89% hydrolyzed, viscosity of 3.4-4.6 mPa·s, was purchased from EMD Chemicals Inc. (480 South Democrat Road Gibbstown, N.J. 08027), product code 1.41350.1001. Cellgro Phosphate-buffered saline 1× (PBS 1×) was purchased from Corning (9345 Discovery Blvd. Manassas, Va. 20109), product code 21-040-CV. Sodium cholate hydrate was purchased from Sigma-Aldrich, (3050 Spruce St. St. Louis, Mo. 63103), part number C6445.

### Method

Solutions were prepared as follows:

Solution 1: M362 was prepared at 40 mg per mL with 100 mg/mL of sodium cholate hydrate in E-free water. Solution 2: PLGA was prepared by dissolving PLGA at 100 mg per 1 mL of dichloromethane in the chemical fume hood. Solution 3: PLGA-PEG was prepared by dissolving PLGA-PEG at 100 mg per 1 mL of dichloromethane in the chemical fume hood. Solution 4: Polyvinyl alcohol was prepared at 50 mg/mL in 100 mM phosphate buffer, pH 8.

A primary (W1/O) emulsion was first created by mixing Solutions 1 through 3. Solution 1 (0.2 mL), Solution 2 (0.50 mL), and Solution 3 (0.50 mL) were combined in a small glass pressure tube pre-chilled for >4 minutes on an ice water bath, and mixed by repeated pipetting. The mixture was then sonicated at 50% amplitude for 40 seconds over an ice bath using a Branson Digital Sonifier 250. A secondary (W1/O/W2) emulsion was then formed by adding Solution 4 (3 mL) to the primary emulsion, vortex mixed, and then sonicated at 30% amplitude for 60 seconds over an ice bath using the Branson Digital Sonifier 250. The secondary emulsion was added to an open 50 mL beaker containing PBS 1× (30 mL). The emulsion was stirred at room temperature for 2 hours to allow the dichloromethane to evaporate and the nanocarriers to form in suspension. A portion of the nanocarrier suspension was washed by transferring the nanocarrier suspension to a centrifuge tubes, spinning at 75,600 rcf for 50 minutes, removing the supernatant, and re-suspending the pellet in PBS 1×. This washing procedure was repeated and then the pellet was re-suspended in PBS 1× to achieve a nanocarrier suspension having a nominal concentration of 10 mg/mL on a polymer basis. The washed nanocarrier suspension was filtered and stored frozen at −20° C.

Size of the nanocarriers was measured by dynamic light scattering. Nanocarrier yield was measured using a gravimetric method. The M362 content was measured using a quantitative assay.

| | Effective | M362 CpG | Nanocarrier |
|---|---|---|---|
| Nanocarrier ID | Diameter (nm) | Content (% w/w) | Yield (%) |
| | 138 | 4.25 | 85 |

### Example 2

### Synthetic Nanocarrier Formulation

### Materials

Synthetic oligonucleotide M362 CpG (5′-tcgtcgtcgttc:gaacgacgttgat-3′), (M362), was purchased from Oligo Factory (120 Jeffrey Aye, Holliston, Mass. 01746), custom manufacture number M362/Selecta Biosciences Lot Number: 1557. Poly(lactide-co-glycolide) polymer, (PLGA), with 54% lactide and 46% glycolide content and an inherent viscosity of 0.24 dL/g was purchased from Lakeshore Biomaterials (756 Tom Martin Drive, Birmingham, Ala. 35211), product code 5050 DLG 2.5 A. Polylactide-bl-Poly(ethylene glycol) block co-polymer with a methyl ether terminated PEG block of approximately 5,000 Da and Mw of 48,000 Da, inherent viscosity of 0.50 dL/g (PLA-PEG-OMe), was purchased from Lakeshore Biomaterials (756 Tom Martin Drive, Birmingham, Ala. 35211), product code 100 DL mPEG 5000 SCE. EMPROVE® Polyvinyl Alcohol 4-88, USP, 85-89% hydrolyzed, viscosity of 3.4-4.6 mPa·s, was purchased from EMD Chemicals Inc. (480 South Democrat Road Gibbstown, N.J. 08027), product code 1.41350.1001. Cellgro Phosphate-buffered saline 1× (PBS 1×) was purchased from Corning (9345 Discovery Blvd. Manassas, Va. 20109), product code 21-040-CV. Sodium cholate hydrate was purchased from Sigma-Aldrich, (3050 Spruce St. St. Louis, Mo. 63103), part number C6445.

### Method

Solutions were prepared as follows:

Solution 1: M362 oligonucleotide was prepared at 40 mg per mL with 100 mg/mL of sodium cholate hydrate in E-free water. Solution 2: PLGA was prepared by dissolving PLGA at 100 mg per 1 mL of dichloromethane in the chemical fume hood. Solution 3: PLA-PEG-OMe was prepared by dissolving PLA-PEG-OMe at 100 mg per 1 mL of dichloromethane in the chemical fume hood. Solution 4: Polyvinyl alcohol was prepared at 50 mg/mL in 100 mM phosphate buffer, pH 8.

A primary (W1/O) emulsion was first created by mixing Solutions 1 through 3. Solution 1 (0.2 mL), Solution 2 (0.50 mL), and Solution 3 (0.50 mL) were combined in a small glass pressure tube pre-chilled for >4 minutes on an ice water bath, and mixed by repeated pipetting. The mixture was then sonicated at 50% amplitude for 40 seconds over an ice bath using a Branson Digital Sonifier 250. A secondary (W1/O/W2) emulsion was then formed by adding Solution 4 (3 mL) to the primary emulsion, vortex mixed, and then sonicated at 30% amplitude for 60 seconds over an ice bath using the Branson Digital Sonifier 250. The secondary emulsion was added to an open 50 mL beaker containing PBS 1× (30 mL). The emulsion was stirred at room temperature for 2 hours to allow the dichloromethane to evaporate and the nanocarriers to form in suspension. A portion of the nanocarrier suspension was washed by transferring the nanocarrier suspension to a centrifuge tubes, spinning at 75,600 rcf for 50 minutes, removing the supernatant, and re-suspending the pellet in PBS 1×. This washing procedure was repeated and then the pellet was re-suspended in PBS 1× to achieve a nanocarrier suspension having a nominal concentration of 10 mg/mL on a polymer basis. The washed nanocarrier suspension was filtered and stored frozen at −20° C.

Size of the nanocarriers was measured by dynamic light scattering. Nanocarrier yield was measured using a gravimetric method. The M362 content was measured using a quantitative assay.

| | Effective | M362 Content | Nanocarrier |
|---|---|---|---|
| Nanocarrier ID | Diameter (nm) | (% w/w) | Yield (%) |
| | 151 | 6.17 | 85 |

### Example 3

### Synthetic Nanocarrier Formulation-Polymer Only Control

### Materials

Poly(lactide-co-glycolide), (PLGA), with 54% lactide and 46% glycolide content and an inherent viscosity of 0.24 dL/g was purchased from Lakeshore Biomaterials (756 Tom Martin Drive, Birmingham, Ala. 35211), product code 5050 DLG 2.5 A). Poly(lactide)-bl-Poly(ethylene glycol), block copolymer with a methyl ether terminated PEG block of approximately 5,000 Da and Mw of 28,000 Da (PLA-PEG-OMe), inherent viscosity of 0.38 dL/g was purchased from Lakeshore Biomaterials (756 Tom Martin Drive, Birmingham, Ala. 35211), product code 100 DL mPEG 5000 4CE. EMPROVE® Polyvinyl Alcohol 4-88, USP, 85-89% hydrolyzed, viscosity of 3.4-4.6 mPa, was purchased from EMD Chemicals Inc. (480 South Democrat Road Gibbstown, N.J. 08027), product code 1.41350.1001. Cellgro Phosphate-buffered saline 1× (PBS 1×) was purchased from Corning (9345 Discovery Blvd. Manassas, Va. 20109), product code 21-040-CV.

### Method

Solutions were prepared as follows:

Solution 1: PLGA was prepared by dissolving PLGA at 100 mg per 1 mL of dichloromethane. Solution 2: PLA-PEG-OMe was prepared by dissolving PLA-PEG-OMe at 100 mg per 1 mL of dichloromethane. Solution 3: Polyvinyl alcohol was prepared at 75 mg/mL in 100 mM phosphate buffer, pH 8.

A single emulsion (W/O) was created by mixing Solutions 1, 2, and 3. Solution 1 (0.75 mL), and Solution 2 (0.25 mL), and Solution 3 (3.0 mL) were added to a small glass pressure tube, vortex mixed, and then emulsified by sonication at 30% amplitude for 60 seconds using a Branson Digital Sonifier 250. The emulsion was added to an open 50 mL beaker containing PBS 1× (30 mL). A second, identical formulation was prepared as above and added to a separate 50 mL beaker containing PBS 1× (30 mL). The two formulations were stirred at room temperature for 2 hours to allow the dichloromethane to evaporate and the nanocarriers to form in suspension. A portion of each nanocarrier suspension was washed by transferring them to separate centrifuge tubes, spinning at 75,600 rcf for 50 minutes, removing the supernatant, and re-suspending the pellets in PBS 1×. This washing procedure was repeated and then the pellets were re-suspended in PBS 1× to achieve nanocarrier suspensions having a nominal concentration of 10 mg/mL on a polymer basis. Each nanocarrier suspension was sterile filtered, then both were pooled together and vortex mixed. The filtered, pooled suspension was stored frozen at −20° C. Nanocarrier yield was determined using a gravimetric method. Size was determined using dynamic light scattering.

| | | |
|---|---|---|
| | Effective Diameter | Nanocarrier Yield |
| Nanocarrier ID | (nm) | (%) |

| | |
|---|---|
| 156.4 | 78 |

### Example 4

### Synthetic Nanocarrier Formulation-Polymer Only Control

### Materials

PLGA with 54% lactide and 46% glycolide content and an inherent viscosity of 0.24 dL/g was purchased from Lakeshore Biomaterials (756 Tom Martin Drive, Birmingham, Ala. 35211), product code 5050 DLG 2.5 A). Polylactide-bl-Poly(ethylene glycol) block co-polymer with a methyl ether terminated PEG block of approximately 5,000 Da and Mw of 28,000 Da (PLA-PEG-OMe), inherent viscosity of 0.38 dL/g was purchased from Lakeshore Biomaterials (756 Tom Martin Drive, Birmingham, Ala. 35211), product code 100 DL mPEG 5000 4CE. EMPROVE® Polyvinyl Alcohol 4-88, USP, 85-89% hydrolyzed, viscosity of 3.4-4.6 mPa·s, was purchased from EMD Chemicals Inc. (480 South Democrat Road Gibbstown, N.J. 08027), product code 1.41350.1001. Cellgro Phosphate-buffered saline 1× (PBS 1×) was purchased from Corning (9345 Discovery Blvd. Manassas, Va. 20109), product code 21-040-CV.

### Method

Solutions were prepared as follows:

Solution 1: PLGA was prepared by dissolving PLGA at 100 mg per 1 mL of dichloromethane in the chemical fume hood. Solution 2: PLA-PEG-OMe was prepared by dissolving PLA-PEG-OMe at 100 mg per 1 mL of dichloromethane in the chemical fume hood. Solution 3: Polyvinyl alcohol was prepared at 50 mg/mL in 100 mM phosphate buffer, pH 8.

An emulsion was formed by mixing Solutions 1 through 3. Solution 1 (0.75 mL), Solution 2 (0.25 mL), and Solution 3 (3.0 mL) were combined in a small glass pressure tube, and vortex mixed. The crude emulsion was then sonicated at 30% amplitude for 60 seconds using a Branson Digital Sonifier 250. The emulsion was stirred at room temperature for 2 hours to allow the dichloromethane to evaporate and the nanocarriers to form in suspension. A portion of the suspended nanocarriers was washed by transferring the nanocarrier suspension to a centrifuge tube, spinning at 75,600 rcf for 50 minutes, removing the supernatant, and re-suspending the pellet in PBS 1×. This washing procedure was repeated and then the pellet was re-suspended in PBS 1× to achieve a nanocarrier suspension having a nominal concentration of 10 mg/mL on a polymer basis. The nanocarrier suspension was filtered and stored frozen at −20° C.

Size of the nanocarriers was measured by dynamic light scattering. Nanocarrier yield was measured using a gravimetric method.

| | | |
|---|---|---|
| | Effective Diameter | Nanocarrier Yield |
| Nanocarrier ID | (nm) | (%) |

| | |
|---|---|
| 201 | 86 |

### Example 5

### Synthetic Nanocarrier Formulation

### Materials

Synthetic peptide from the human papilloma virus HPV-16 E7 protein, residues 49-57 (HPV peptide), was prepared by Peptides International Inc., (11621 Electron Drive Louisville, Ky. 40299). PLGA with 54% lactide and 46% glycolide content and an inherent viscosity of 0.24 dL/g was purchased from Lakeshore Biomaterials (756 Tom Martin Drive, Birmingham, Ala. 35211), product code 5050 DLG 2.5 A). PLA-PEG block co-polymer with a methyl ether terminated PEG block of approximately 5,000 Da and Mw of 28,000 Da, inherent viscosity of 0.38 dL/g was purchased from Lakeshore Biomaterials (756 Tom Martin Drive, Birmingham, Ala. 35211), product code 100 DL mPEG 5000 4CE. EMPROVE® Polyvinyl Alcohol 4-88, USP, 85-89% hydrolyzed, viscosity of 3.4-4.6 mPa·s, was purchased from EMD Chemicals Inc. (480 South Democrat Road Gibbstown, N.J. 08027), product code 1.41350.1001. Cellgro Phosphate-buffered saline 1× (PBS 1×) was purchased from Corning (9345 Discovery Blvd. Manassas, Va. 20109), product code 21-040-CV.

### Method

Solutions were prepared as follows:

Solution 1: HPV peptide was prepared at 10 mg per mL in 0.13M hydrochloric acid with 10% by volume formamide. Solution 2: PLGA was prepared by dissolving PLGA at 100 mg per 1 mL of dichloromethane in the chemical fume hood. Solution 3: PLA-PEG-OMe was prepared by dissolving PLA-PEG-OMe at 100 mg per 1 mL of dichloromethane in the chemical fume hood. Solution 4: Polyvinyl alcohol was prepared at 50 mg/mL in 100 mM phosphate buffer, pH 8.

A primary (W1/O) emulsion was first created by mixing Solutions 1 through 3. Solution 1 (0.2 mL), Solution 2 (0.75 mL), and Solution 3 (0.25 mL) were combined in a small glass pressure tube, and mixed by repeated pipetting. The pressure tube was then held for 4 minutes in an ice water bath, and sonicated at 50% amplitude for 40 seconds over an ice bath using a Branson Digital Sonifier 250. A secondary (W1/O/W2) emulsion was then formed by adding Solution 4 (3 mL) to the primary emulsion, vortex mixed, and then sonicated at 30% amplitude for 60 seconds over an ice bath using the Branson Digital Sonifier 250. The secondary emulsion was added to an open 50 mL beaker containing PBS 1× (30 mL). A second identical double emulsion formulation was prepared as described above, and added to the same 50 mL beaker as the first. The combined emulsions were stirred at room temperature for 2 hours to allow the dichloromethane to evaporate and the nanocarriers to form in suspension. A portion of the suspended nanocarriers was washed by transferring the nanocarrier suspension to a centrifuge tube, spinning at 75,600 rcf for 50 minutes, removing the supernatant, and re-suspending the pellet in PBS 1×. This washing procedure was repeated and then the pellet was re-suspended in PBS 1× to achieve a nanocarrier suspension having a nominal concentration of 10 mg/mL on a polymer basis. The nanocarrier suspension was sterile filtered, and stored frozen at −20° C.

Size of the nanocarriers was measured by dynamic light scattering. Nanocarrier yield was measured using a gravimetric method. The HPV peptide content was measured using a quantitative assay.

| | Effective | HPV Peptide | Nanocarrier |
|---|---|---|---|
| Nanocarrier ID | Diameter (nm) | Content (% w/w) | Yield (%) |
| | 148 | 0.86 | 87 |

### Example 6

### Synthetic Nanocarrier Formulation

### Materials

Synthetic peptide from the human papilloma virus HPV-16 E7 protein, residues 49-57 (HPV peptide), was prepared by Peptides International Inc., (11621 Electron Drive Louisville, Ky. 40299). PLGA with 54% lactide and 46% glycolide content and an inherent viscosity of 0.24 dL/g was purchased from Lakeshore Biomaterials (756 Tom Martin Drive, Birmingham, Ala. 35211), product code 5050 DLG 2.5 A). Polylactide-bl-Poly(ethylene glycol) block co-polymer with a methyl ether terminated PEG block of approximately 5,000 Da and Mw of 28,000 Da (PLA-PEG-OMe), inherent viscosity of 0.38 dL/g was purchased from Lakeshore Biomaterials (756 Tom Martin Drive, Birmingham, Ala. 35211), product code 100 DL mPEG 5000 4CE. EMPROVE® Polyvinyl Alcohol 4-88, USP, 85-89% hydrolyzed, viscosity of 3.4-4.6 mPa·s, was purchased from EMD Chemicals Inc. (480 South Democrat Road Gibbstown, N.J. 08027), product code 1.41350.1001. Cellgro Phosphate-buffered saline 1× (PBS 1×) was purchased from Corning (9345 Discovery Blvd. Manassas, Va. 20109), product code 21-040-CV.

### Method

Solutions were prepared as follows:

Solution 1: HPV peptide was prepared at 10 mg per mL in 0.13M hydrochloric acid with 10% by volume formamide. Solution 2: PLGA was prepared by dissolving PLGA at 100 mg per 1 mL of dichloromethane in the chemical fume hood. Solution 3: PLA-PEG-OMe was prepared by dissolving PLA-PEG-OMe at 100 mg per 1 mL of dichloromethane in the chemical fume hood. Solution 4: Polyvinyl alcohol was prepared at 50 mg/mL in 100 mM phosphate buffer, pH 8.

A primary (W1/O) emulsion was first created by mixing Solutions 1 through 3. Solution 1 (0.2 mL), Solution 2 (0.75 mL), and Solution 3 (0.25 mL) were combined in a small glass pressure tube pre-chilled for four minutes in an ice water bath, and mixed by repeated pipetting. The crude emulsion was then sonicated at 50% amplitude for 40 seconds over an ice bath using a Branson Digital Sonifier 250. A secondary (W1/O/W2) emulsion was then formed by adding Solution 4 (3 mL) to the primary emulsion, vortex mixed, and then sonicated at 30% amplitude for 60 seconds over an ice bath using the Branson Digital Sonifier 250. The secondary emulsion was added to an open 50 mL beaker containing PBS 1× (30 mL). A second identical double emulsion formulation was prepared as described above, and added to the same 50 mL beaker as the first. An additional set of double emulsions were prepared and added to a separate 50 mL beaker with PBS 1× (30 mL), as the first. The emulsions were stirred at room temperature for 2 hours to allow the dichloromethane to evaporate and the nanocarriers to form in suspension. A portion of the suspended nanocarriers were washed by transferring the nanocarrier suspension to centrifuge tubes, spinning at 75,600 rcf for 50 minutes, removing the supernatant, and re-suspending the pellets in PBS 1×. This washing procedure was repeated and then the pellets were re-suspended in PBS 1× to achieve a nanocarrier suspensions having a nominal concentration of 10 mg/mL on a polymer basis. The nanocarrier suspensions were sterile filtered, pooled together, and stored frozen at −20° C.

Size was measured by dynamic light scattering. Nanocarrier yield was measured using a gravimetric method. The HPV peptide content was measured using a quantitative assay.

| | Effective | HPV Peptide | Nanocarrier |
|---|---|---|---|
| Nanocarrier ID | Diameter (nm) | Content (% w/w) | Yield (%) |
| | 148 | 0.80 | 72 |

### Example 7

### Synthetic Nanocarrier Formulation

### Materials

TRP2 synthetic peptide sequence SVYDFFVWL, (TRP2), was custom synthesized by Peptides International Inc., (11621 Electron Drive Louisville, Ky. 40299) catalog number PCS-37153-PI, lot number 001639C. Poly(lactide-co-glycolide) polymer, (PLGA), with 54% lactide and 46% glycolide content and an inherent viscosity of 0.24 dL/g was purchased from Lakeshore Biomaterials (756 Tom Martin Drive, Birmingham, Ala. 35211), product code 5050 DLG 2.5 A. Polylactide-bl-poly(ethylene glycol) block co-polymer with a methyl ether terminated PEG block of approximately 5,000 Da and Mw of 48,000 Da, inherent viscosity of 0.50 dL/g (PLA-PEG-OMe), was purchased from Lakeshore Biomaterials (756 Tom Martin Drive, Birmingham, Ala. 35211), product code 100 DL mPEG 5000 SCE. EMPROVE® Polyvinyl Alcohol 4-88, USP, 85-89% hydrolyzed, viscosity of 3.4-4.6 mPa·s, was purchased from EMD Chemicals Inc. (480 South Democrat Road Gibbstown, N.J. 08027), product code 1.41350.1001. Cellgro Phosphate-buffered saline 1× (PBS 1×) was purchased from Corning (9345 Discovery Blvd. Manassas, Va. 20109), product code 21-040-CV. Sucrose was purchased from Sigma-Aldrich (3050 Spruce St. St. Louis, Mo. 63103), product code S9378-1KG.

### Method

Solutions were prepared as follows:

Solution 1: TRP2 was prepared at 20 mg per 1 mL solution containing 0.1M sodium hydroxide and 125 mg sucrose per 1 mL solution. Solution 2: PLGA was prepared by dissolving PLGA at 100 mg per 1 mL of dichloromethane. Solution 3: PLA-PEG-OMe was prepared by dissolving PLA-PEG-OMe at 100 mg per 1 mL of dichloromethane. Solution 4: Polyvinyl alcohol was prepared at 50 mg/mL in 100 mM phosphate buffer, pH 8.

A primary (W1/O) emulsion was first created by mixing Solutions 1 through 3. Solution 1 (0.2 mL), Solution 2 (0.75 mL), and Solution 3 (0.25 mL) were combined in a small glass pressure tube, mixed by repeated pipetting, and held in an ice water bath for four minutes. The mixture was then sonicated at 50% amplitude for 40 seconds over an ice bath using a Branson Digital Sonifier 250. A secondary (W1/O/W2) emulsion was then formed by adding Solution 4 (3 mL) to the primary emulsion, vortex mixed, and then sonicated at 30% amplitude for 60 seconds over an ice bath using the Branson Digital Sonifier 250. The secondary emulsion was added to an open 50 mL beaker containing PBS 1× (30 mL). The emulsion was stirred at room temperature for 2 hours to allow the dichloromethane to evaporate and the nanocarriers to form in suspension. A portion of the nanocarrier suspension was washed by transferring the nanocarrier suspension to a centrifuge tube, spinning at 75,600 rcf for 50 minutes, removing the supernatant, and re-suspending the pellet in PBS 1×. This washing procedure was repeated and then the pellet was re-suspended in PBS 1× to achieve a nanocarrier suspension having a nominal concentration of 10 mg/mL on a polymer basis. The nanocarrier suspension was filtered and stored frozen at −20° C.

Size was measured by dynamic light scattering. Nanocarrier yield was measured using a gravimetric method. The TRP2 content was measured using a quantitative assay.

| | Effective | TRP2 Content | Nanocarrier |
|---|---|---|---|
| Nanocarrier ID | Diameter (nm) | (% w/w) | Yield (%) |
| | 160 | 0.88 | 70 |

### Example 8

### Evaluating Effects of Administering Synthetic Nanocarriers and Immune Checkpoint Inhibitors

Synthetic nanocarrier compositions comprising a dominant E7 peptide epitope, E7.I.49 (SVP-E7.I.49) and TLR9 agonist type C CpG oligonucleotide M362 (SVP-M362) were prepared as described in the above Examples. C57BL/6 age-matched mice were injected subscapularly with 0.5×10⁵ cells of the canercous cell line, TC-1, which expressed the E7 oncogene from human papilloma virus 16 (HPV-16). The mice were therapeutically treated with the synthetic nanocarriers comprising the E7 peptide epitope and CpG oligonucleotide (SVP-E7.I.49+SVP-M362) or empty synthetic nanocarriers (SVP-empty) on days 6, 10, 17, 24 after tumor inoculation via subscapular route. Three different groups of experimental animals were either left without additional treatment or co-treated with an anti-PD-L1 antibody (10F.9G2, BioXCell, Catalog # BE0101) or an isotype control antibody via intraperitoneal route, following the initial administration of nanocarriers on days 11, 14, and 18 after tumor inoculation. The tumor burden (volume, mm²) and percent survival of the experimental animals was assessed throughout the course of the experiment.

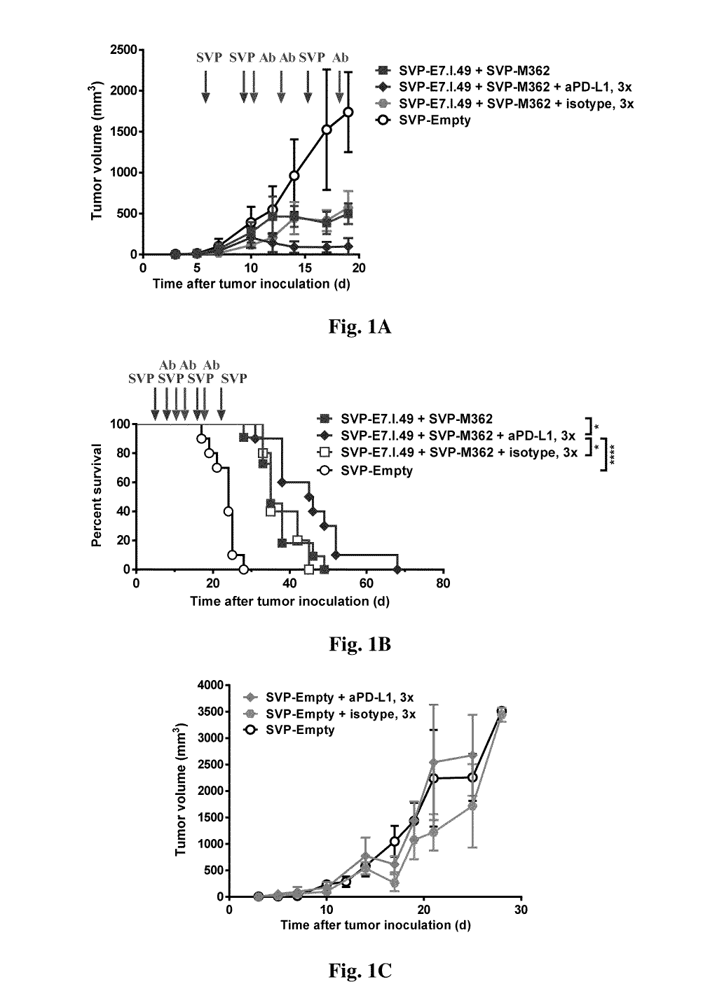

While administration of the synthetic nanocarrier compositions comprising the E7 peptide epitope and CpG oligonucleotide (SVP-E7.I.49+SVP-M-362) resulted in a substantially lower tumor burden, this effect was further enhanced by co-treatment with the anti-PD-L1 antibody, but not with the isotype control antibody (FIG. 1A). Animals that received the synthetic nanocarrier compositions comprising the E7 peptide epitope and CpG oligonucleotide (SVP-E7.I.49+SVP-M-362) also survived longer than animals that received empty synthetic nanocarriers (FIG. 1B). The overall survival was also enhanced by cotreatment with the anti-PD-L1 antibody, but not with the isotype control antibody.

An additional set of C57BL/6 age-matched mice were injected subscapularly with 0.5×10⁵ cells of the cancercous cell line, TC-1, then treated with compositions of empty synthetic nanocarriers alone or in combination with the anti-PD-L1 antibody or an isotype control antibody, administered three times. No therapeutic effect was observed in the absence of the synthetic nanocarriers comprising the antigen and immunostimulator (FIG. 1C).

These results indicate that the immune checkpoint inhibitor and synthetic nanocarrier compositions functioned synergistically to enhance therapeutic effects of the anti-tumor treatment.

### Example 9

### Evaluating Effects of Administering Synthetic Nanocarriers and Immune Checkpoint Inhibitors

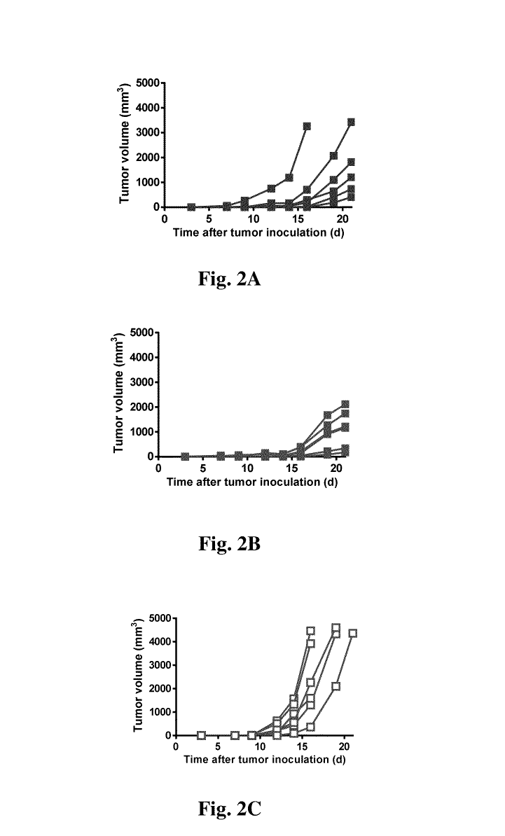

Synthetic nanocarrier compositions comprising a dominant tumor-specific peptide epitopes from tyrosinase-related protein 2 (TRP2) and TLR7/8 agonist R848 were prepared. C57BL/6 age-matched mice were injected subscapularly with 0.5×10⁵ cells of the mouse melanoma line B16-F10. One group of mice was therapeutically treated with the synthetic nanocarriers comprising the TRP2 peptide epitope and R848 on days 1, 4, 11, and 18 via subcapsular route, and the tumor burden (volume, mm²) was assessed over the course of the experiment (FIG. 2A). In addition to the treatment with the synthetic nanocarriers comprising the TRP2 peptide epitope and R848 on days 1, 3, 4, 11, and 18, a second group of mice was co-treated with an anti-PD-L1 antibody (10F.9G2, BioXCell, Catalog # BE0101) after the initial administration of the synthetic nanocarriers. The tumor burden (volume, mm²) of these animals was also assessed over the course of the experiment (FIG. 2B). A third group of mice received the anti-PD-L1 antibody on days 2, 6, and 9 via intraperitoneal route without administration of the synthetic nanocarrier compositions (FIG. 2C).

While administration of the synthetic nanocarrier compositions comprising the TRP2 peptide epitope and R848 resulted in a substantially lower tumor burden as compared to the tumor burden in mice that only received the anti-PD-L1 antibody without the synthetic nanocarriers (FIGS. 2A and 2C), this effect was further enhanced by co-treatment of the synthetic nanocarriers comprising the TRP2 peptide epitope and R848 with the anti-PD-L1 antibody (FIG. 2B).

These results indicate that the immune checkpoint inhibitor and synthetic nanocarrier compositions functioned synergistically to enhance therapeutic effects of the anti-tumor treatment.

### Example 10

### Attaching Nanocarrier to R848 Adjuvant Abolishes Systemic Production of Inflammatory Cytokines

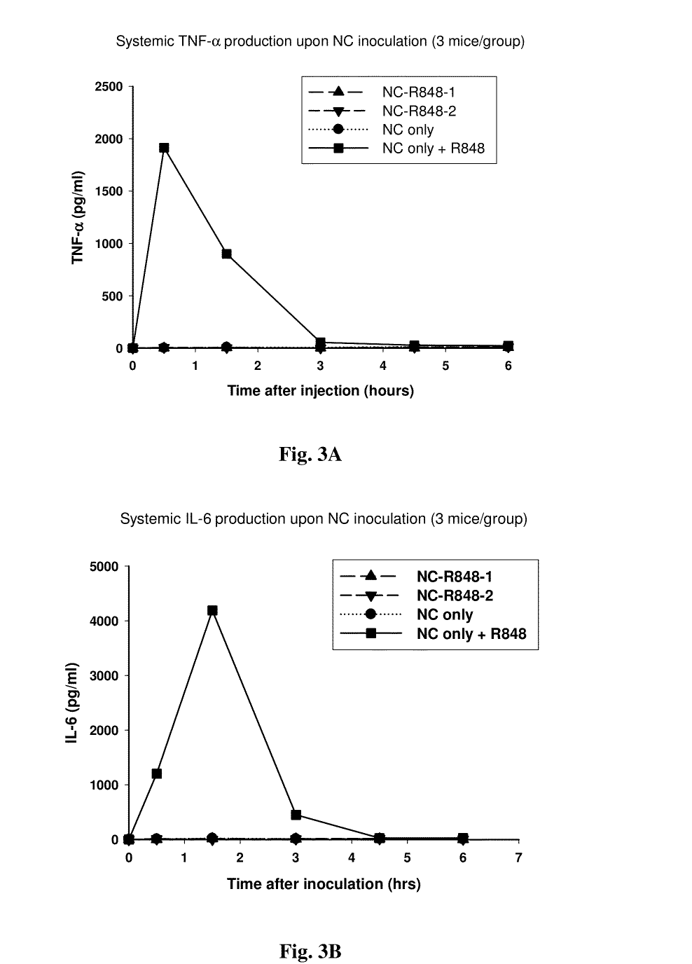

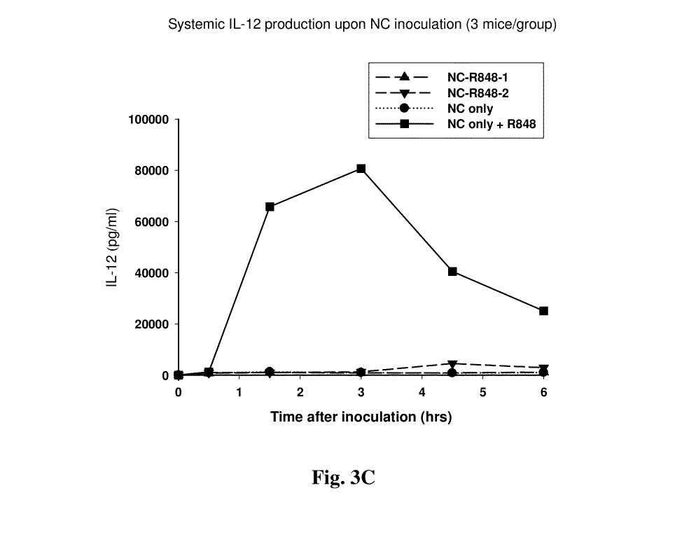

Nanocarrier compositions were prepared as described in U.S. Publication No. 20120027806. Groups of mice were injected subcutaneously into hind limbs with 100 μg of nanocarriers (NC) coupled, non-coupled or admixed with small molecule nucleoside analogue and known TLR7/8 agonist and adjuvant R848. R848 amount in nanocarrier was 2-3% resulting in 2-3 μg of coupled R848 per injection; amount of free R848 used was 20 μg per injection. Mouse serum was taken by terminal bleed and systemic cytokine production in serum was measured at different time-points by ELISA (BD Biosciences). As seen in FIGS. 3A-3C, strong systemic production of major pro-inflammatory cytokines TNF-α, IL-6 and IL-12 was observed when admixed R848 (NC+R848) was used, while no expression of TNF-α, IL-6 and IL-12 was detected when two separate preparations of NC coupled with R848 (NC-R848-1 and NC-R848-2) were used.

The difference in peak cytokine expression levels was >100-fold for TNF-α and IL-6, and >50-fold for IL-12. NC not coupled to R848 (labeled as NC only) did not induce any systemic cytokines when used without admixed R848.

### Example 11

### Nanocarriers with Entrapped Adjuvant Result in Lower Systemic Proinflammatory Cytokine Induction

### Materials for Nanocarrier Formulations

Ovalbumin peptide 323-339 amide acetate salt, was purchased from Bachem Americas Inc. (3132 Kashiwa Street, Torrance Calif. 90505. Product code 4065609.) PS-1826 DNA oligonucleotide with fully phosphorothioated backbone having nucleotide sequence 5′-TCC ATG ACG TTC CTG ACG TT-3′ with a sodium counter-ion was purchased from Oligos Etc (9775 SW Commerce Circle C-6, Wilsonville, Oreg. 97070.) PLA with an inherent viscosity of 0.19 dL/g was purchased from Boehringer Ingelheim (Ingelheim Germany. Product Code R202H). PLA-PEG-Nicotine with a nicotine-terminated PEG block of approximately 5,000 Da and DL-PLA block of approximately 17,000 Da was synthesized. Polyvinyl alcohol (Mw=11,000-31,000, 87-89% hydrolyzed) was purchased from J.T. Baker (Part Number U232-08).

### Methods for Nanocarrier Production

Solutions were prepared as follows:

Solution 1: Ovalbumin peptide 323-339 @ 70 mg/mL in dilute hydrochloric acid aqueous solution. The solution was prepared by dissolving ovalbumin peptide in 0.13N hydrochloric acid solution at room temperature.

Solution 2: 0.19-IV PLA @ 75 mg/mL and PLA-PEG-nicotine @ 25 mg/ml in dichloromethane. The solution was prepared by separately dissolving PLA @ 100 mg/mL in dichloromethane and PLA-PEG-nicotine @ 100 mg/mL in dichloromethane, then mixing the solutions by adding 3 parts PLA solution for each part of PLA-PEG-nicotine solution.

Solution 3: Oligonucleotide (PS-1826) @ 200 mg/ml in purified water. The solution was prepared by dissolving oligonucleotide in purified water at room temperature.

Solution 4: Same as solution 2.

Solution 5: Polyvinyl alcohol @ 50 mg/mL in 100 mM pH 8 phosphate buffer.

Two separate primary water in oil emulsions were prepared. W1/O was prepared by combining solution 1 (0.1 mL) and solution 2 (1.0 mL) in a small pressure tube and sonicating at 50% amplitude for 40 seconds using a Branson Digital Sonifier 250. W3/O4 was prepared by combining solution 3 (0.1 mL) and solution 4 (1.0 mL) in a small pressure tube and sonicating at 50% amplitude for 40 seconds using a Branson Digital Sonifier 250. A third emulsion with two inner emulsion phases ([W1/O2,W3/O4]/W5) emulsion was prepared by combining 0.5 ml of each primary emulsion (W1/O2 and W3/O4) and solution 5 (3.0 mL) and sonicating at 30% amplitude for 60 seconds using the Branson Digital Sonifier 250.

The third emulsion was added to an open 50 mL beaker containing 70 mM pH 8 phosphate buffer solution (30 mL) and stirred at room temperature for 2 hours to evaporate dichloromethane and to form nanocarriers in aqueous suspension. A portion of the nanocarriers was washed by transferring the suspension to a centrifuge tube and spinning at 13,800 g for one hour, removing the supernatant, and re-suspending the pellet in phosphate buffered saline. The washing procedure was repeated and the pellet was re-suspended in phosphate buffered saline for a final nanocarrier dispersion of about 10 mg/mL.

The amounts of oligonucleotide and peptide in the nanocarrier were determined by HPLC analysis. The total dry-nanocarrier mass per mL of suspension was determined by a gravimetric method and was adjusted to 5 mg/mL. Particles were stored as refrigerated suspensions until use.

### Nanocarrier Characterization

| | Effective | TLR Agonist, | T-cell helper |
|---|---|---|---|
| Nanocarrier | Diameter (nm) | % w/w | peptide, % w/w |
| | 232 | PS-1826, 6.4 | Ova, 2.2 |

### Results

TNF-α and IL-6 were induced in sera of NC-CpG- and free CpG-inoculated animals. Animal groups were inoculated (s.c.) either with 100 μg of NC-CpG (containing 5% CpG-1826) or with 5 μg of free CpG-1826. At different time-points post inoculation serum was collected from the animals (3/group) by terminal bleed, pooled and assayed for cytokine presence in ELISA (BD).

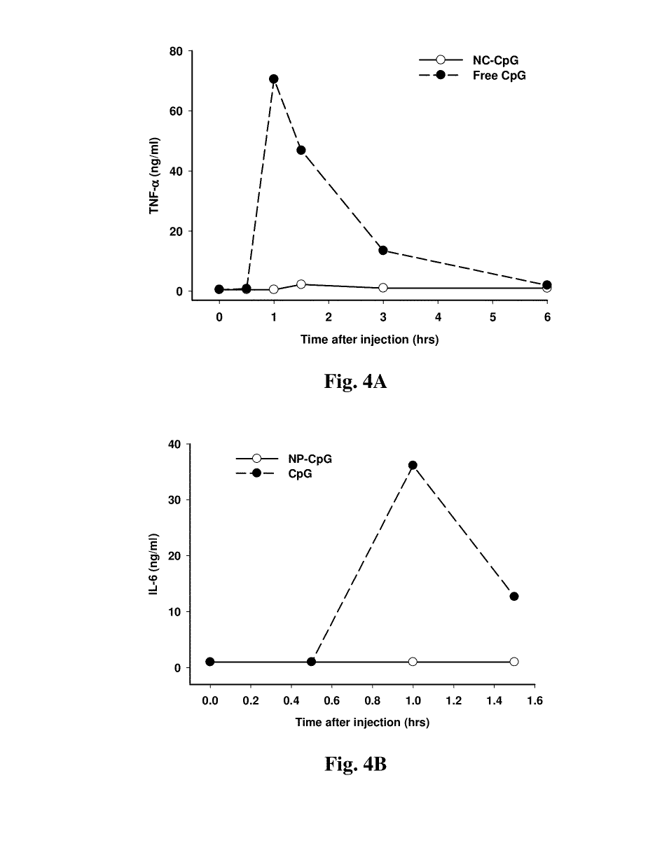

The results demonstrate that entrapment of adjuvant within NC results in a lower immediate systemic proinflammatory cytokine induction than utilization of free adjuvant. When identical amounts of a CpG adjuvant, NC-entrapped or free, were used for inoculation, a substantially higher induction of TNF-α and IL-6 in animal serum was observed for free CpG compared to NC-entrapped CpG (FIG. 4).

1. A method comprising:
providing or obtaining a synthetic nanocarrier composition comprising a first population of synthetic nanocarriers that are attached to an antigen and a second population of synthetic nanocarriers that are attached to an immunostimulator;
providing or obtaining a composition comprising an immune checkpoint inhibitor; and
administering the synthetic nanocarrier composition and immune checkpoint inhibitor composition to a subject.

2. The method of claim 1, wherein the first population of synthetic nanocarriers and the second population of synthetic nanocarriers are the same population of synthetic nanocarriers.

3. The method of claim 1, wherein the first population of synthetic nanocarriers and the second population of synthetic nanocarriers are different populations of synthetic nanocarriers.

4. The method of claim 1, wherein the antigen and immunostimulator are encapsulated within the synthetic nanocarriers of the synthetic nanocarrier composition.

5. The method of claim 1, wherein the synthetic nanocarrier composition and immune checkpoint inhibitor composition are administered concomitantly to the subject.

6. The method of claim 1, wherein the synthetic nanocarrier composition is administered prior to the immune checkpoint inhibitor composition.

7. The method of claim 1, wherein the synthetic nanocarrier composition is administered at least four times to the subject and the immune checkpoint inhibitor composition of administered at least three times to the subject.

8. The method of claim 1, wherein the synthetic nanocarrier composition and immune checkpoint inhibitor composition are each administered five times to the subject.

9. The method of claim 1, wherein the synthetic nanocarrier composition and immune checkpoint inhibitor composition are administered to a subject according to a protocol that has been shown to result in an enhanced immune response against the antigen.

10. The method of claim 1, wherein the synthetic nanocarrier composition and immune checkpoint inhibitor composition are administered to a subject according to a protocol that has been shown to result in a reduced immunosuppressive immune response against the antigen.

11. The method of claim 1, wherein the method further comprises determining the protocol.

12. The method of claim 1, wherein the subject has or is at risk of having cancer or an infection or infectious disease.

13. The method of claim 1, wherein the method further comprises assessing an immune response against the antigen in the subject prior to, during or subsequent to administration of the synthetic nanocarrier composition or immune checkpoint inhibitor composition.

14. The method of claim 1, wherein the administering is by intravenous, intraperitoneal or subcutaneous administration.

15. A composition or kit comprising:
a synthetic nanocarrier dose, wherein the synthetic nanocarrier dose comprises a first population of synthetic nanocarriers that are attached to an antigen and a second population of synthetic nanocarriers that are attached to an immunostimulator; and
a dose of an immune checkpoint inhibitor composition.

16. The composition or kit of claim 15, wherein the composition or kit is for use in any one of the methods provided herein.

17. The composition or kit of claim 15, further comprising a pharmaceutically acceptable carrier.

18. The composition or kit of claim 1, wherein the antigen and immunostimulator are encapsulated within the synthetic nanocarriers of the synthetic nanocarrier composition.

19. The composition or kit of claim 1, wherein the first population of synthetic nanocarriers and the second population of synthetic nanocarriers are the same population of synthetic nanocarriers.

20. The composition or kit of claim 1, wherein the first population of synthetic nanocarriers and the second population of synthetic nanocarriesr are different populations of synthetic nanocarriers.

21-75. (canceled)
